SEQUENCE LISTING (38 sequences)

SEQ ID NO: 1 - protein, 915 aa, Neisseria gonorrhoeae
MQQQHLFRLNILCLSLMTALPAYAENVQAGQAQEKQLDTIQVKAKKQKTRRDNEVTGLGKLVKTADTLSKEQVLDIRDLTRYDPGIAVVEQGRGASSGYSIRGMDKNRVSLTVDGLAQIQSYTAQAALGGTRTAGSSGAINEIEYENVKAVEISKGSNSVEQGSGALAGSVAFQTKTADDVIGEGRQWGIQSKTAYSGKNRGLTQSIALAGRIGGAEALLIRTGRHAGEIRAHEAAGRGVQSFNRLAPVDDGSKYAYFIVEEECKNGGHEKCKANPKKDVVGEDKRQTVSTRDYTGPNRFLADPLSYESRSWLFRPGFRFENKRHYIGGILERTQQTFDTRDMTVPAFLTKAVFDANQKQAGSLRGNGKYAGNHKYGGLFTSGENNAPVGAEYGTGVFYDETHTKSRYGLEYVYTNADKDTWADYARLSYDRQGIGLDNHFQQTHCSADGSDKYCRPSADKPFSYYKSDRVIYGESHKLLQAAFKKSFDTAKIRHNLSVNLGYDRFGSNLRHQDYYYQSANRAYSLKTPPQNNGKKTSPNGREKNPYWVSIGRGNVVTRQICLFGNNTYTDCTPRSINGKSYYAAVRDNVRLGRWADVGAGLRYDYRSTHSDDGSVSTGTHRTLSWNAGIVLKPADWLDLTYRTSTGFRLPSFAEMYGWRSGDKIKAVKIDPEKSFNKEAGIVFKGDFGNLEASWFNNAYRDLIVRGYEAQIKDGKEQVKGNPAYLNAQSARITGINILGKIDWNGVWDKLPEGWYSTFAYNRVRVRDIKKRADRTDIQSHLFDAIQPSRYVVGSGYDQPEGKWGVNGMLTYSKAKEITELLGSRALLNGNSRNTKATARRTRPWYIVDVSGYYTVKKHFTLRAGVYNLLNHRYVTWENVRQTAAGAVNQHKNVGVYNRYAAPGRNYTFSLEMKF

SEQ ID NO: 2 - DNA, 2745 bp, Neisseria gonorrhoeae
atgcaacagcaacatttgttccgattaaatattttatgcctgtctttaatgactgcgctgcccgcttatgcagaaaatgtgcaagccggacaagcacaggaaaaacagttggataccatacaggtaaaagccaaaaaacagaaaacccgccgcgataacgaagtaaccggtttgggcaaattggtcaaaaccgccgacaccctcagcaaggaacaggtactcgacatccgcgacctgacgcgttacgaccccggcatcgccgtcgtcgaacaggggcgcggcgcaagctcgggctactcgatacgcggtatggacaaaaaccgcgtctccttgacggtggacggcttggcgcaaatacagtcctacaccgcgcaggcggcattgggcgggacgaggacggcgggcagcagcggcgcaatcaatgaaatcgagtatgagaacgtcaaggctgtcgaaatcagcaaaggctcaaactcggtcgaacaaggcagcggcgcattggcgggctcggtcgcatttcaaaccaaaaccgccgacgatgttatcggggaaggcaggcagtggggcattcagagtaaaaccgcctattccggcaaaaaccgggggcttacccaaaggtagcgcgaccgccccgcgtagccgccacgcctccgaaacgactaggcgtggcccgcccacgcgggggaaatccgcgcccacgaagccgccggacgcggcgttcagagcttcaacaggctggcgccggttgatgacggcagcaagtacgcctatttcatcgttgaagaagaatgcaaaaacgggggtcacgaaaagtgtaaagcgaatccgaaaaaagatgttgtcggcgaagacaaacgtcaaacggtttccacccgagactacacgggccccaaccgcttcctcgccgatccgctttcatacgaaagccggtcgtggctgttccgcccgggttttcgttttgaaaacaaacggcactacatcggcggcatactcgaacgcacgcaacaaactttcgacacgcgcgatatgacggttccggcatttctgaccaaggcggtttttgatgcaaatcaaaaacaggcgggttctttgcgcggcaacggcaaatacgcgggcaaccacaaatacggcggactgtttaccagcggcgaaaacaatgcgccggtgggcgcggaatacggtacgggcgtgttttacgacgagacgcacaccaaaagccgctacggtttggaatatgtctataccaatgccgataaagacacttgggcggattatgcccgcctctcttacgaccggcagggcatcggtttggacaaccattttcagcagacgcactgttccgccgacggttcggacaaatattgccgtccgagtgccgacaagccgttttcctattacaaatccgaccgcgtgatttacggggaaagccataagctcttgcaggcggcattcaaaaaatccttcgataccgccaaaatccgccacaacctgagcgtgaatctcggttacgaccgcttcggctctaatctccgccatcaggattattattatcaaagtgccaaccgcgcctattcgttgaaaacgccccctcaaaacaacggcaaaaaaaccagccccaacggcagagaaaagaatccctattgggtcagcataggcaggggaaatgtcgttacgaggcaaatctgcctctttggcaacaatacttatacggactgcacgccgcgcagcatcaacggcaaaagctattacgcggcggtccgggacaatgtccgtttgggcaggtgggcggatgtcggcgcgggcttgcgctacgactaccgcagcacgcattcggacgacggcagcgtttccaccggcacgcaccgcaccctgtcctggaacgccggcatcgtcctcaaacctgccgactggctggatttgacttaccgcacttcaaccggcttccgcctgccctcgtttgcggaaatgtacggctggcggtcgggcgataaaataaaagccgtcaaaatcgatccggaaaaatcgttcaacaaagaagccggcatcgtgtttaaaggcgatttcggcaacttggaggcaagttggttcaacaatgcctaccgcgatttgattgtccggggttatgaagcgcaaattaaagacggcaaagaacaagtcaaaggcaacccggcttacctcaatgcccaaagcgcgcggattaccggcatcaatattttgggcaaaatcgattggaacggcgtatgggataaattgcccgaaggttggtattccacatttgcctataatcgtgtccgtgtccgcgacatcaaaaaacgcgcagaccgcaccgatattcaatcacacctgtttgatgccatccaaccctcgcgctatgtcgtcggctcgggctatgaccaaccggaaggcaaatggggcgtgaacggtatgctgacttattccaaagccaaggaaatcacagagttgttgggcagccgggctttgctcaacggcaacagccgcaatacaaaagccaccgcgcgccgtacccgcccttggtatattgtggacgtgtccggttattacacggttaaaaaacacttcaccctccgtgcgggcgtgtacaacctcctcaaccaccgctatgttacttgggaaaatgtgcggcaaactgccgccggcgcagtcaaccaacacaaaaatgtcggcgtttacaaccgatatgccgcccccggccgcaactacacatttagcttggaaatgaagttc

SEQ ID NO: 3 - protein, 728 aa, Neisseria gonorrhoeae
MNNPLVNQAAMVLPVFLLSACLGGGGSFDLDSVDTEAPRPAPKYQDVPSKKPEARKDQGGYGFAMRFKRRNWHPSANPKEDEVKLKNDDWEATGLPTEPKKLPLKQQSVISEVETNGNSKMYTSPYLSQDADSSHANGANQPKNEVTDYKKFKYVYSGWFYKHAKSEVKNENGLVSAKRGDDGYIFYHGDKPSRQLPASEDDGYIFYHGDKPSRQLPASEAVTYKGVWHFVTDTKQGQKFNDILETSKGQGDKYSGFSGDEGETTSNRTDSNLNDKHEGYGFTSNFKVDFNNKKLTGKLIRNNKVINTAASDGYTTEYYSLDATLRGNRFSGKAIATDKPNTGGTKLHPFVFDSSSLSGGFFGPQGEELGFRFLSDDGKVAVVGSAKTKDSTANGNAPAASSGPGAATMPSETRLTTVLDAVELTPDGKEIKNLDNFSNATRLVVDGIMIPLLPTESGNGQADKGKNGGTDFTYETTYTPESDKKDTKAQTGAGGMQTASGTAGVNGGQVGTKTYKVQVCCSNLNYLKYGLLTRENNNSVMQAVKNSSQADAKTKQIEQSMFLQGERTDENKIPQEQGIVYLGFWYGRIANGTSWSGKASNATDGNRAKFTVNFDRKEITGTLTAENRSEATFTIDAMIEGNGFKGTAKTGNDGFAPDQNNSTVTHKVHIANAEVQGGFYGPNAEELGGWFAYPGNEQTKNATVESGNGNSASSATVVFGAKRQKLVK

SEQ ID NO: 4 - DNA, 2187 bp, Neisseria gonorrhoeae
atgaacaatccattggtgaatcaggctgctatggtgctgcccgtgtttttgttgagcgcttgtctgggcggaggcggcagtttcgatcttgattctgtcgataccgaagccccgcgtcccgcgccaaagtatcaagatgttccttccaaaaaaccggaagcccgaaaagaccaaggcggatacggttttgcaatgcgcttcaagcggcggaattggcatccgagtgcgaatcctaaagaagatgaggttaaattaaagaatgatgattgggaggcgacaggattgccgacagaacccaagaaactgccattaaaacaacaatccgtcatttcagaagtagaaaccaacggtaattctaaaatgtacacttcaccttatctcagtcaagatgcagatagtagtcatgcaaatggtgcaaaccaaccaaaaaacgaagtaacagattacaaaaaattcaaatatgtttattccggctggttttacaaacacgcgaaaagcgaagtcaaaaacgaaaacggattagtaagtgcaaaaagaggcgatgacggctatatcttttatcacggcgacaaaccttcccgacaacttcccgcttctgaagatgacggctatatcttttatcacggcgacaaaccttcccgacaacttcccgcttctgaagcagttacctataaaggtgtgtggcattttgtaaccgatacgaaacagggacaaaaatttaacgatattcttgaaacctcaaaagggcaaggcgacaaatacagcggattttcgggcgatgaaggcgaaacaacttccaatagaactgattccaaccttaatgataagcacgagggttatggttttacctcaaattttaaagtggatttcaataataaaaaattgacgggcaaactgattcgcaacaataaagttataaacactgctgctagcgacggatataccaccgaatattacagtctcgatgcgacgcttaggggaaaccgcttcagcggcaaggcgatagcgaccgacaaacccaacactggaggaaccaaactacatccctttgttttcgactcgtcttctttgagcggcggctttttcggcccgcagggtgaggaattgggtttccgctttttgagcgacgatggaaaagttgccgttgtcggcagcgcgaaaaccaaagacagcaccgcaaatggcaatgctccggcggcttcaagcggcccaggtgcggcaactatgccgtctgaaaccaggctgaccacggttttggatgcggttgaattgacaccagacggcaaggaaatcaaaaatctcgacaacttcagcaacgctacccgactggttgtcgacggcattatgattccgctcctgcccaccgaaagcgggaacggtcaggcagataaaggtaaaaacggcggaacagactttacctacgaaacaacctacacgccggaaagtgataaaaaagacaccaaagcccaaacaggcgcgggcggcatgcaaaccgcttcgggtacggcgggcgttaacggcgggcaggtaggaacaaaaacctataaagtccaagtctgctgttccaacctcaattatctgaaatacgggctgctgacacgtgaaaacaacaattccgtgatgcaggcagtcaaaaacagtagtcaagctgatgctaaaacgaaacaaattgaacaaagtatgttcctccaaggcgagcgcaccgatgaaaacaagattccacaagagcaaggcatcgtttatctggggttttggtacgggcgtattgccaacggcacaagctggagcggcaaggcttccaatgcaacggatggcaacagggcgaaatttaccgtgaatttcgataggaaagaaattaccggcacgttaaccgctgaaaacaggtcggaggcaacctttaccattgacgccatgattgagggcaacggctttaaaggtacggcgaaaaccggtaatgacggatttgcgccggatcaaaacaatagcaccgttacacataaagtgcacatcgcaaatgccgaagtgcagggcggtttttacgggcctaacgccgaagagttgggcggatggtttgcctatccgggcaatgaacaaacgaaaaatgcaacagttgaatccggcaatggaaattcagcaagcagtgcaactgtcgtattcggtgcgaaacgccaaaagcttgtgaaataa

SEQ ID NO: 5 - protein, 707 aa, Neisseria gonorrhoeae
LGGGGSFDLDSVDTEAPRPAPKYQDVPSKKPEARKDQGGYGFAMRFKRRNWHPSANPKEDEVKLKNDDWEATGLPTEPKKLPLKQQSVISEVETNGNSKMYTSPYLSQDADSSHANGANQPKNEVTDYKKFKYVYSGWFYKHAKSEVKNENGLVSAKRGDDGYIFYHGDKPSRQLPASEDDGYIFYHGDKPSRQLPASEAVTYKGVWHFVTDTKQGQKFNDILETSKGQGDKYSGFSGDEGETTSNRTDSNLNDKHEGYGFTSNFKVDFNNKKLTGKLIRNNKVINTAASDGYTTEYYSLDATLRGNRFSGKAIATDKPNTGGTKLHPFVFDSSSLSGGFFGPQGEELGFRFLSDDGKVAVVGSAKTKDSTANGNAPAASSGPGAATMPSETRLTTVLDAVELTPDGKEIKNLDNFSNATRLVVDGIMIPLLPTESGNGQADKGKNGGTDFTYETTYTPESDKKDTKAQTGAGGMQTASGTAGVNGGQVGTKTYKVQVCCSNLNYLKYGLLTRENNNSVMQAVKNSSQADAKTKQIEQSMFLQGERTDENKIPQEQGIVYLGFWYGRIANGTSWSGKASNATDGNRAKFTVNFDRKEITGTLTAENRSEATFTIDAMIEGNGFKGTAKTGNDGFAPDQNNSTVTHKVHIANAEVQGGFYGPNAEELGGWFAYPGNEQTKNATVESGNGNSASSATVVFGAKRQKLVK

SEQ ID NO: 6 - protein, 405 aa, Artificial
  NB domain
LGGGGSFDLDSVDTEAPRPAPKYQDVPSKKPEARKDQGGYGFAMRFKRRNWHPSANPKEDEVKLKNDDWEATGLPTEPKKLPLKQQSVISEVETNGNSKMYTSPYLSQDADSSHANGANQPKNEVTDYKKFKYVYSGWFYKHAKSEVKNENGLVSAKRGDDGYIFYHGDKPSRQLPASEAVTYKGVWHFVTDTKQGQKFNDILETSKGQGDKYSGFSGDEGETTSNRTDSNLNDKHEGYGFTSNFKVDFNNKKLTGKLIRNNKVINTAASDGYTTEYYSLDATLRGNRFSGKAIATDKPNTGGTKLHPFVFDSSSLSGGFFGPQGEELGFRFLSDDGKVAVVGSAKTKDSTANGNAPAASSGPGAATMPSETRLTTVLDAVELTPDGKEIKNLDNFSNATRLVVD

SEQ ID NO: 7 - protein, 83 aa, Artificial
  L2 domain
RTGRHAGEIRAHEAAGRGVQSFNRLAPVDDGSKYAYFIVEEECKNGGHEKCKANPKKDVVGEDKRQTVSTRDYTGPNRFLADP

SEQ ID NO: 8 - protein, 492 aa, Artificial
  L2/NB fusion protein
LGGGGSFDLDSVDTEAPRPAPKYQDVPSKKPEARKDQGGYGFAMRFKRRNWHPSANPKEDEVKLKNDDWEATGLPTEPKKLPLKQQSVISEVETNGNSKMYTSPYLSQDADSSHANGANQPKNEVTDYKKFKYVYSGWFYKHAKSEVKNENGLVSAKRGDDGYIFYHGDKPSRQLPASEAVTYKGVWHFVTDTKQGQKFNDILETSKGQGDKYSGFSGDEGETTSNRTDSNLNDKHEGYGFTSNFKVDFNNKKLTGKLIRNNKVINTAASDGYTTEYYSLDATLRGNRFSGKAIATDKPNTGGTKLHPFVFDSSSLSGGFFGPQGEELGFRFLSDDGKVAVVGSAKTKDSTANGNAPAASSGPGAATMPSETRLTTVLDAVELTPDGKEIKNLDNFSNATRLVVDLEGSRTGRHAGEIRAHEAAGRGVQSFNRLAPVDDGSKYAYFIVEEECKNGGHEKCKANPKKDVVGEDKRQTVSTRDYTGPNRFLADP

SEQ ID NO: 9 - protein, 702 aa, Neisseria gonorrhoeae
MNNPLVNQAAMVLPVFLLSACLGGGGSFDLDSVDTEAPRPAPKYQDVPSKKPEARKDQGGYGFAMRFKRRNWYPPSNPKENEIRLSEGDWEQTGNGNIKNPSKQKNIIDALSGNGEAPLQDSSQQGRGISKVTDYHDFKYVWSGFFYKQIGNTIKKDDSSSKIIEARNGPDGYIFYKGTDPSRKLPVSGSVEYKGTWDFLTDVQANQKFTDLGSAFTKSGDRYSAFSGELDYIVRKEEDKKDGEVGLALTTEITVNFEKKTLSGKLIKNNMVINNGDKPTTQYYSLEAQVTGNRFNGKAMVTDKPENSKSKQHPFVSDSSSLSGGFFGPQGEELGFRFLSNDKVAVVGSAKTKDETASSGGTSGGASVSASNGATGTSSGNSNLTTVIDAVELTPDGKEIKDLDNFSNAAQLVVDGIMIPLLSTESGNGQADKGKNGGTDFTYTTTYMPESDKKDTKAQTGAGGMQTASDAAGVNGGQAGTKTYKVEACCSNLNYLKYGLLTRENSNSVLQTVRNSSQAAARTAQGAQSMFLQGERTDEKEIPKEQKVVYLGTWYGHIAANGTSWTGNASDQQSGNRAKEDVNFKDKKTTGTLTAANRQEATFTIDAMIDDNGFKGTAKTGNDGFAPDQNSSTGTYKVHIANAEVQGGFYGPNAEELGGWFAYPGNGQTKNAQENAQASSGNGNSAVSATVVFGAKRQQLVK

SEQ ID NO: 10 - protein, 683 aa, Neisseria gonorrhoeae
MNNPLVNQAAMVLPVFLLSACLGGGGSFDLDSVDTEAPRAAPKYQDVPSKKPEARKDQGGYGFAMRFKRRNWYRAANENEVKLKESDWEQTDDDEIKNPPKQKNIINALPGNEGELLQDSSQQGKGTSKVRDHHDFKYVWSGFFYKRIKITTKKDESHKIIEARSGPDGYIFYKGRNPSRKLPVSGEVTYKGTWDFLTDVKANQKFTDLGNASTKSGDQYSAFSGELDYIVKKEEDKKSKHKGLGLTTEITVDFEKKTLIGKLIKNNMIINNNTKPTTQYYSLEAQVTGNRFSGKAMATEKGENKQHPFVSDSSSLSGGFFGPQGEELGFRFLSDDGKVAVVGSAKTKDETASSGGTSGGASVSTSNGAAGTSSENKLTTVLDAVELTPNGKKIKDLDNFSNAAQLVVDGIMIPLLPKDSESGGSHTDKGENGKTAFIYETTYTPESDKEDAQTGMATNGVQTVSNTAGGTSGKTKTKYRVQACCSNLNYLKYGLLTRKNSSQADAKMGQVEQSMFLQGERTDEKEIPQEQNVVYSGTWYGHIATNGTSWTREASDQENGNRANFDVNFKDKRTTGTLTAENRSEATFTIEAMIEGNGFKGTAKTGNGGFAPDQNSSTGTKKVHITNAAVQGGFYGPNAEELGGWFAYPGNGQTKNAQTSSGNGNSAGSATVVFGAKRQQLVK

SEQ ID NO: 11 - protein, 689 aa, Neisseria gonorrhoeae
MNNPLVNQAAMVLPVFLLSACLGGGGSFDLDSVDTEAPRPAPKYQDVPSKKPEARKHQGGYGFAMRFKRRNWNRAANENEVKLNESNWERTDDGDIKKPSKQQNIIDALTGNDGETLQDSNQEGQSISKVEGEHDFKYVWSGFFYKHIQTKHETIDGKITVRSGPDGYIFYKGKDPSRKLPVSGKVMYKGTWDFLTDVKANQRFTDLGNTSARSGDQYSAFSGELDYIVRKEEDKKDGRVGNGLTTEITVDFEKKTLNGKLIKNNRVINDNDEHTTQYYSLEAQVTGNRFNGKAIATDKPKANETKEHPFVSDSPSLSGGFFGPKGEELVFRFLSDDGKVAVVGSAKTRDKAANGNTASASGDTGAARMPSETGLTTVLDAVELTLDGKEIKNLDNFSDATRLVVDGIMIPLLSTESGNGQADKGKNGGTDFIYETTYMPESDKKDTQTGMAADGVQTVSNAAGGTSGKTKTYKVQVCCSNLNYLKYGLLTRENNNSVMQAGESSSRTAAQTQGAQSMFLQGERTDEKEIPKDENVVYLGSWYGYIANGTSWSGNASNTTSGNKANFTVNFDKKKTTGMLTAANRQEATFTIDAMIESNGFKGMAKTGNQGFAPDQNSSTGTKKVRIAEAKVRGGFYGPNAEELGGWFAYPGNGQTKNAQENAQASSGNGNSAGSATVVFGAKRQELVK

SEQ ID NO: 12 - protein, 709 aa, Neisseria gonorrhoeae
MNNPLVNQAAMVLPVFLLSACLGGGGSFDLDSVDTEAPRPAPKYQDVPSKKPEARKDQGGYGFAMRFKRRNWHPGANPKEDRVKLKNDDWEATGLPTEPKKLPLKQQSVISEVETNGNSKMYTSPYLSQDADSSHANGANQPKNEVTDYKKFKYVYSGWFYKHAKSEVKNENGEVSAKRGDDGYIFYHGDKPSRQLPASEAVTYKGVWHFVTDTKQGQKFNDILETSKGQGDRYSGFSGDEGETTSNRTDSKLNDKHEGYGFTSNFKVDFNNKKLTGKLIRNNIKVINTAASDGYTTEYYSLDATLRGNRFSGKAIATDKPNTGGTKLHPFVFDSSSLSGGFFGPQGEELGFRFLSDDGKVAVVGSAKTKDSTANGNAPAASSGPGAATMPSETRLTTVLDAVELTPDGKEIKNLDNFSNATRLVVDGIMIPLLPTESGNGQADKGKNGGTDFTYETTYTPESDKKDTKAQTGAGGMQTASGTAGVNGGQVGTKTYKVQVCCSNLNYLKYGNLTRENNNSVMQAVKNSSQADAKTKQIEQSMFLQGERTDENKIPQEQGIVYLGFWYGRIANGTSWSGKASNATDGNRAKFTVNFDRKEITGTLTAENRSEATFTIDAMIEGNGFKGTAKTGNDGFAPDQNNSTVTHKVHIANAEVQGGFYGPNAEELGGWFAYPGNEQTKNATVESGNGNSASSATVVFGAKRQELVK

SEQ ID NO: 13 - protein, 707 aa, Neisseria gonorrhoeae
MNNPLVSQAAMVLPVFLLSACLGGGGSFDLDSVDTEAPRPAPKYQDVPSEKPEARKDQGGYGFAMRFKRRNRNPMAIPKENEVKLKDDDWEATGLPGDPKDLPGRQKSVIDEVSANDNNDIYFSPYLKPSNHQNSSINGSANQPKNEVTDYKNFKYVYSGWFYKHAKPIIDGIQNKDQQGDDGYIFYHGNKPSRQLPASEAVTYKGVWHFVTDTKQGQKFNDILEASKGQGDRYSGFSGDEGETTSNRTDSKLNDKHEGYGFTSNFKVDFNNKKLTGKLIRNNKVINTAASDGYTTEYYSLDATLRGNRFSGKAMATDKPNTGGTKLHPFVSDSSSLSGGFFGPQGEELGFRFLSDDGKVAVVGSAKTRDKANNGNTASASGDTGAAAMPSETRLTTVLDAVELTPNGKEIKNLDNFSNAARLVVDGIMIPLLPTESGNGQADKGKNGGTAFTYTTTYTPESDKKDTKAQTGAGGMQTASDAAGVNGGQAGTKTYEVKACCSNLNYLKYGLLTRKTAGNTGEGGKGSQAAAQTAQGAQSMFLQGERTDEKEIPKDGDVVYLGTWYGGIANGTSWTREASNQENGNRAKFDVNFKDKRITGTLTAENRSEATFTIDGEIEGNGFKGTAKTGNDGFAPDQNNSTVTHKVRIANAEVQGGFYGPNAEELGGWFAYPGNEQTKNAQASSGNGNSAGSATVVFGAKRQELVQ

SEQ ID NO: 14 - protein, 693 aa, Neisseria meningitidis
CLGGGGFFDLDSVDTEAPRPDPKYQDVSSEKPQAQKDQGGYGFAMRLKRRNWYSAAKEDEVKLNESDWETTGLPTEPKKLPLKQESVISKVQANNGDNNIYTSPYLTQSNHQNSSINGGANLPKNKVTNYKNFKYVYSGWFYKHAKNKIIRENSSIKGAKNGDDGYIFYHGKEPSRQLPASETVTYKGVWHFATDVKKSQNFRDIIQPSKKQGDRYSGFSGDDDEQYSNKNESMLKDGQEGYGFTSNLEVDFGSKKLTGKLIRNNKVTNAPTNDKYTTQYYSLDAQITGNRFNGKAIRTDKPDTGGTKLHPFVSDSSSLSGGFFGPKGEELGFRFLSDDKKVAVVGSAKTRDKTENGAVASGGTDAAASNGAAGTSSENSKLTTVLDAVELKLGDKEVQKLDNFSNAAQLVVDGIMIPLLPEASESGNNQANQGTNGGTAFTRKFDHTPESDKKDAQAGTQTNGAQTASNTAGDTNGKTKTYEVEVCCSNLNYLKYGMLTRKNSKSAMQAGESSSQADAKTEQVEQSMFLQGERTDEKEIPSEQNIVYRGSWYGYIANDKSTSWSGNASNATSGNRAEFTVNEADKKITGTLTADNRQEATFTIDGNIKDNGFEGTAKTAESGFDLDQSNTTRTPKAYITDAKVQGGFYGPNAEELGGWFAYPGDKQTKNATNASGNSSATVVFGAKRQQPVR

SEQ ID NO: 15 - protein, 711 aa, Neisseria meningitidis
MNNPLVNQAAMVLPVFLLSACLGGGGSFDLDSVDTEAPRPAPKYQDVSSEKPQAQKDQGGYGFAMRLKRRNWYPGAEESEVKLNESDWEATGLPTKPKELPKRQKSVIEKVETDSDSDIYSSPYLTPSNHQNGSAGNGVNQPKNQATGKENFQYVYSGWFYKHAASEKDFSNKKIKSGDDGYIFYHGKKPSRQLPASGKVIYKGVWHFVTDTKKGQDFREIIQPSKKQGDRYSGFSGDGSEEYSNKNESTLKDDHEGYGFTSNLEVDFGNKKLTGKLIRNNASLNNNTNNDKHTTQYYSLDAQITGNRFNGTATATDKKENETKLHPFVSDSSSLSGGFFGPQGEELGFRFLSDDQKVAVVGSAKTKDKLENGAAASGSTGAAASGGAAGTSSENSKLTTVLDAVELTLNDKKIKNLDNFSNAAQLVVDGIMIPLLPKDSESGNTQADKGKNGGTEFTRKFEHTPESDKKDAQAGTQTNGAQTASNTAGDTNGKTKTYEVEVCCSNLNYLKYGMLTRKNSKSAMQAGGNSSQADAKTEQVEQSMFLQGERTDEKEIPTDQNVVYRGSWYGHIANGTSWSGNASDKEGGNRAEFTVNEADKKITGKLTAENRQAQTFTIEGMIQGNGFEGTAKTAESGFDLDQKNTTRTPKAYITDAKVKGGFYGPNAEELGGWFAYPGDKQTKKATATSSDGNSASSATVVFGAKRQQPVQ

SEQ ID NO: 16 - protein, 699 aa, Neisseria meningitidis
CLGGGGGSFDLDSVDTEAPRPAPKYQDVSSEKPQAQKDQGGYGFAMRLKRRNWYPSAKENEVKLNESDWETTGLPSNPKNLPKRQKSVIEQVETDGDSNNSNIYSSPYLTQSNHQNGNTGNGVNQPKNEVTDYKNFKYVYSGWFYKHAKREVNLAVEPKIAKNGDDGYIFYHGKDPSRQLPASGKITYKGVWHFATDTKKGQKFREIIQPSKNQGDRYSGFSGDDDSQYSNKNESMLKDGHEGYGFASNLEVDFDNKKLTGKLIRNNANQNNNTNNDKHTTQYYSLDATLKGNRFSGKAEATDKPKNDGETKEHPFVSDSSSLSGGFFGPQGEELGFRFLSNDQKVAVVGSAKTKDKPANGNTAEASGSTDAAASGGAAGTSSENSKLTTVLDAVELTHGGTAIKNLDNFSNAAQLVVDGIMIPLLPQNSTGKNNQPDQGKNGGTAFIYKTTYTPKNDDKDTKAQTVTGGTQTASNTAGDANGKTKTYEVEVCCSNLNYLKYGLLTRKTAGNTVGSGNSSPTAAAQTDAQSMFLQGERTDENKIPSEQNVVYRGSWYGHIASSTSWSGNASDKEGGNRAEFTVNEGEKKITGTLTAENRQEATFTIDGKIEGNGFSGTAKTAELGFDLDQKNTTRTPKAYITDAKVKGGFYGPNAEELGGWFAYSDDKQTKKATDASSNGNSASSATVVFGAKRQQPVQ

SEQ ID NO: 17 - protein, 706 aa, Neisseria meningitidis
CLGGGGGFFDLDSVDTEAPRPAPKYQDVSSEKPQAQKDQGGYGFAMRLKRRNWHPQANPKEDEIKLSENDWEATGLPGNPKNLPERQKSVIEKVKTGSDSNIYSSPYLTQSNHQNGSANQPKNEVKDYKEFKYVYSGWFYKHAKLEIILENNLIKGAKSGDDGYIFYHGEKPSRQLPVSGEVTYKGVWHFVTDTKQGQKFNDILGTSKKQGDRYSGFPGDDGEEYSNKNEATLQGSQEGYGFTSNLEVDFNKKKLTGELIRNNRVINATANDKYTTQYYSLEAQVTGNRFNGKATATDKPGTGETKQHPFVSDSSSLSGGFFGPKGEELGFRFLSNDQKVAVVGSAKTQDKAANGNTAAASGGTDAAASNGAAGTSSENSKLTTVLDAVELTLNDKKIKNLDNFSNAAQLVVDGIMIPLLPETSESGSNQADKGKKGKNGKNGGTADTYKTTYTPKNDDKDTKAQTGAASSSGAQTDLGKADVNGGKAETKTYEVEVCCSNLNYLKYGLLTRKNSKSAMQAGGNSSQADAKTEQVEQSMFLQGERTDEKEIPNDQNVVYRGSWYGRIASSTSWSGNASNATSGNRAEFTVNEDTKKINGTLTAENRQEATFTIDGKIEGNGFSGTAKTADLGFDLDQSNTTGTPKAYITDAKVQGGFYGPNAEELGGWFAYPGDKQTEKATVASSDGNSASSATVVFGAKRQQPVQ

SEQ ID NO: 18 - protein, 601 aa, Neisseria meningitidis
MNNPLVNQAANVLPVFLLSACLGGGGGSFDLDSVETVQDMHSKPKYEDEKSQPESQQDVSENSGAAYGFAVKLPRRNAHFNPKYKEKHKPLGSMDWKKLQRGRPNSFSERDELEKKRGSSELIESKWEDGQSRVVGYTNFTYVRSGYVYLNKNNIDIKNNIVLFGPDGYIYYKGKEPSKELPSEKITYKGTWDYVTDAMEKQRFEGLGSAAGGDRSGALSALKKGVLRNQAEASSSGETDFGMTSEFEVDFSDKTLKGTLYRNNRITQNNSENKQIKTTRYTIQATLHGNRFKGKALAADKGATNGSHPFISDSCSLEGGFYGPKGEELAGKFLSNDNKVAAVFGAKQKDKKDGENAAGPATETVIDAYRITGEEFKKEQIDSFGDVKKLVVDGVELSLLPSEGNKAAFQHEIEQNGVKATVCCSNLDYMSFGKLSKEKKDDMFLQGVRTPVSDVAARTEANAKYRGTWYGYLANGTSWSGEASNQEGGNRAEEDVDESTKKISGTLTAKDRTSPAFTITAMIKDNGFSGVAKTGENGFALDPQNTGNSHYTHIEATMSGGFYGKNAIEMGGSFSFPGNAPSGKQEKASVVFGAKRQQLVQ

SEQ ID NO: 19 - protein, 13 aa, Artificial
  synthetic peptide
QKYADDVIGEGRQ

SEQ ID NO: 20 - protein, 12 aa, Artificial
  synthetic peptide
KKDVVGEDKRQT

SEQ ID NO: 21 - protein, 11 aa, Artificial
  synthetic peptide
RGNGKYAGNHK

SEQ ID NO: 22 - protein, 14 aa, Artificial
  synthetic peptide
KTPPQNNGKKTSPN

SEQ ID NO: 23 - protein, 13 aa, Artificial
  synthetic peptide
HSDDGSVSTGTHR

SEQ ID NO: 24 - protein, 11 aa, Artificial
  synthetic peptide
KDGKEQVKGNP

SEQ ID NO: 25 - protein, 14 aa, Artificial
  synthetic peptide
NSRNTKATARRTRP

SEQ ID NO: 26 - protein, 11 aa, Artificial
  synthetic peptide
PSKKPEARKDQ

SEQ ID NO: 27 - protein, 12 aa, Artificial
  synthetic peptide
NQPKNEVTDYKK

SEQ ID NO: 28 - protein, 11 aa, Artificial
  synthetic peptide
TDTKQGQKFND

SEQ ID NO: 29 - protein, 19 aa, Artificial
  synthetic peptide
DEGETTSNRTDSNLNDKHE

SEQ ID NO: 30 - protein, 16 aa, Artificial
  synthetic peptide
DFNNKKLTGKLIRNNK

SEQ ID NO: 31 - protein, 18 aa, Artificial
  synthetic peptide
TPDEKEIKNLDNFSNATR

SEQ ID NO: 32 - protein, 19 aa, Artificial
  synthetic peptide
TYETTYTPESDKKDTKAQT

SEQ ID NO: 33 - protein, 12 aa, Artificial
  synthetic peptide
KNSSQADAKTKQ

SEQ ID NO: 34 - protein, 14 aa, Artificial
  synthetic peptide
QGERTDENKIPQEQ

SEQ ID NO: 35 - DNA, 31 bp, Artificial
  synthetic oligonucleotide primer
ggatcctgtctgggcggaggcggcagtttcg

SEQ ID NO: 36 - DNA, 32 bp, Artificial
  synthetic oligonucleotide primer
cccgggttatttcacaagcttttggcgtttcg

SEQ ID NO: 37 - DNA, 32 bp, Artificial
  synthetic oligonucleotide primer
tggccacacctcaaaaattactgatttgtgtg

SEQ ID NO: 38 - DNA, 35 bp, Artificial
  synthetic oligonucleotide primer
ctcgagttaatttgccatactaattgcggcaatcg

(19) United States
(12) Patent Application Publication
(10) Pub. No.: US 2008/0206260 A1
(43) Pub. Date: Aug. 28, 2008

(54) Chimeric Tbp-toxin proteins as mucosal adjuvants for vaccination against neisseriae

(76) Inventors: Cynthia Nau Cornelissen, Midlothian, VA (US); Gregory Price, Aurora, CO (US); Michael Russell, Buffalo, NY (US); Terry Connell, Buffalo, NY (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/963,110

(22) Filed: Dec. 21, 2007

Related U.S. Application Data
(63) Continuation-in-part of application No. PCT/US2006/023879, filed on Jun. 20, 2006.
(60) Provisional application No. 60/693,499, filed on Jun. 24, 2005.

(51) Int. Cl. A61K 39/395; C07K 14/00; A61K 39/385; A61K 38/02; A61P 31/04; C07H 21/00; A61K 38/16

(52) U.S. Cl. 424/164.1; 530/394; 424/192.1; 536/23.4; 536/23.7; 514/12; 514/2

(57) ABSTRACT

Chimeric fusion proteins comprising Neisseria transferrin binding proteins (Tbps) from, for example, N. gonorrhoeae and/or N. meningitidis are provided. The fusion proteins elicit an antibody response in the mucosa of the urogenital and/or oropharynx tract, as well as in the serum. The resulting serum antibodies are cross-bactericidal against heterologous bacterial strains. The chimeric proteins also comprise a mucosal adjuvant such as a toxin subunit, e.g. the B subunit of cholera toxin or of Escherichia coli heat labile toxin II. Methods of inhibiting the growth of Neisseria species on mucosal surfaces of a mammal by either administering the fusion proteins of the invention, or antibodies directed to the fusion proteins of the invention, to the mammal.

### CROSS-REFERENCE TO RELATED APPLICATIONS

This application claims benefit of and is a continuation-in-part of International patent application PCT/US2006/023879, filed Jun. 20, 2006, which claims benefit of U.S. provisional patent application 60/693,499, filed Jun. 24, 2005, the complete contents of both of which are hereby incorporated by reference.

### BACKGROUND OF THE INVENTION

1. Field of the Invention

The invention generally relates to chimeric fusion proteins comprising transferrin binding proteins, and their use to elicit an antibody response to Neisseria species in the urogenital and/or oropharynx tract when administered intranasally. In particular, the chimeric proteins comprise Neisseria transferrin binding proteins and a mucosal adjuvant.

2. Background of the Invention

Neisseria gonorrhoeae, the causative agent of the sexually transmitted disease gonorrhea, has been a burden to mankind from antiquity. The earliest documented observations were made by Hippocrates, who lived from 460-355 BC, and references to gonorrhea have been described in biblical passages, as well as other ancient texts from around the world. Central to this bacteria's prolific nature, is its antigenic variability of surface structures, as well as ability to avoid initiating host immune responses.

Only since the post-antibiotic age has control of this infection been possible. However, as with most bacteria under antibiotic pressure, gonococcal antibiotic resistance is increasing. This has resulted in the use of newer and more expensive antibiotics for treating this disease. In developing and third world countries, where gonococcal infection is more rampant, this is a greater problem as newer and more effective antibiotics may not be easily available.

A 1995 WHO report estimated that there were 62.2 million cases of the sexually transmitted infection (STI) gonorrhea world-wide (19). This number is considered to be an underestimate of the actual incidence due in part to inadequate reporting by physicians and clinics, as well as to the prevalence of asymptomatic carriage (8, 40). One study found that asymptomatic carriage in women can be as high as 55% (17). Uncomplicated gonorrhea manifests as urethritis in men and as endocervitis and/or urethritis in women. Serious downstream sequelae can afflict those individuals with asymptomatic infection since the infection can spread to the upper genital tract. Ascension can result in epididimytis, salpingitis, ectopic pregnancy, sterility, and disseminated gonococcal infection.

Antibiotics are the treatment of choice for gonorrhea, but the increasing emergence of drug-resistant strains has made treatment more difficult and expensive (25). Furthermore, it has been shown that co-infection with N. gonorrhoeae and HIV can increase the risk of transmission of the HIV virus (10). These findings have made the need for an effective vaccine more imperative. To date, gonococcal vaccine attempts have been disappointing. Human trials using partially-lysed gonococci, purified pilin, or purified porin all failed to confer protection upon natural exposure (4, 20, 45). These vaccine formulations, although immunogenic, failed to protect, likely due in part to the intrinsic ability of the gonococcus to undergo high frequency phase and antigenic variation of surface structures (28).

The Neisseria transferrin receptor complex is composed of two surface exposed, iron repressible proteins, transferrin binding protein A (ThpA), and transferrin binding protein B (TbpB). ThpA is an integral outer membrane protein, with sequence homology to the TonB-dependent family of outer membrane proteins (Cornelissen, C. N., G. D. Biswas, J. Tsai, D. K. Paruchuri, S. A. Thompson, and P. F. Sparling. 1992. Gonococcal transferrin-binding protein 1 is required for transferrin utilization and is homologous to TonB-dependent outer membrane receptors. J. Bacteriol. 174:5788-5797). It is the larger of the two proteins of the complex, with an approximate molecular weight of 100 kDa. ThpA forms the pore through which iron is shuttled into the bacterial periplasm after it is stripped from transferrin (Anderson, J. E., P. F. Sparling, and C. N. Cornelissen. 1994. Gonococcal transferrin-binding protein 2 facilitates but is not essential for transferrin utilization. J. Bacteriol. 176:3162-70).

TbpB is a lipidated surface exposed protein with a variable molecular weight between 78-86 kDa (Schryvers, A. B., and B. C. Lee. 1989. Comparative analysis of the transferrin and lactoferrin binding proteins in the family Neisseriaceae. Can. J. Microbiol. 35:409-415). TbpB is able to discriminate between iron loaded and apo-transferrin (Cornelissen, C. N., and P. F. Sparling. 1996. Binding and surface exposure characteristics of the gonococcal transferrin receptor are dependent on both transferrin-binding proteins. J. Bacteriol. 178:1437-44). Thus, TbpB is thought to increase the efficiency of this receptor complex by discriminating between iron-loaded and iron-depleted transferrin. Under normal conditions, serum transferrin is only approximately 30% iron saturated (Schryvers, A. B., and I. Stojiljkovic. 1999. Iron acquisition systems in the pathogenic Neisseria. Mol. Microbiol. 32:1117-23.).

Once iron loaded transferrin is bound to ThpA, TbpA is thought to undergo a conformational change allowing ThpA to interact with the TonB complex in the cytoplasmic membrane. In an energy dependent process (12), iron is stripped from transferrin and shuttled into the periplasm through the ThpA pore. The iron then binds to the periplasmic iron binding protein FbpA (Chen, C. Y., S. A. Berish, S. A. Morse, and T. A. Mietzner. 1993. The ferric iron-binding protein of pathogenic Neisseria spp. functions as a periplasmic transport protein in iron acquisition from human transferrin. Mol. Microbiol. 10:311-8), which shuttles it to a cytoplasmic permease. Utilizing energy from ATP, the permease is able to translocate the iron into the cytoplasm.

ThpA and TbpB have generated particular interest as vaccine antigens because they are ubiquitously expressed among clinical isolates, they exhibit low strain-to-strain variability, and they are not subject to high frequency antigenic or phase variation (11, 12, 29). Furthermore, their importance in gonococcal virulence has been established in a human male challenge model of infection (14). Subjects inoculated with a mutant strain of N. gonorrhoeae that lacked the transferrin receptor, showed no signs or symptoms of urethritis, in contrast to subjects inoculated with the parental strain (14). However, in spite of their necessity to establish infection in vivo, antibody responses to the transferrin binding proteins resulting from natural infections are weak in the serum, and non-existent in vaginal washes and seminal fluid (34). It can therefore be postulated that the induction and sustained production of an anti-Tbp antibody response in the genital tract may prevent colonization.

One of the shortcomings of parenteral immunization is its relatively poor ability to induce genital tract specific IgA antibodies (5, 30). IgA is considered important in protecting the genital tract from infection as its presence is correlated with a protective role from chlamydia and HIV (6, 7). Intranasal immunization (IN), on the other hand, has been more promising in terms of eliciting genital tract, antigen-specific IgA and IgG in mice (18, 21, 47), primates (42), and humans (3, 38). In addition, the genital tract antibodies generated as a function of IN immunization have been demonstrated to be long lasting in mice (37, 47)

The prior art has thus far failed to provide compositions and methods for the prevention and treatment of N. gonorrhoeae infection. In particular, the prior art has failed to provide compositions and methods to elicit an IgA or IgG antibody response to N. gonorrhoeae in the genital tract.

Neisseria meningitidis is found in the oropharynx of about 20% of the population without causing any symptoms of disease, i.e. these individuals are carriers of the bacterium. In overcrowded conditions such as military barracks and boarding school dormitories the carriage rate is much higher. However, for reasons that are not understood, the carrier status can break down resulting in the development of bacterial meningococcal meningitis, a disease that is invariably fatal if untreated. The mortality drops to 10% when appropriate therapy is promptly initiated. Penicillin or a third-generation cephalosporin is the treatment of choice, and may be combined with chloramphenicol.

The grouping of meningococci into serogroups is based on the antigenic structure of the bacterial capsule. A vaccine is available that protects against Groups A, C, W-135 and Y, and another that is bivalent and protects only against Groups A and C. Unfortunately, however, the predominant disease-causing strains belong to Group B, for which there is currently no effective vaccine.

The prior art has thus far failed to provide a vaccine against N. meningitidis that is broadly protective against all serotypes of meningococci.

### SUMMARY OF THE INVENTION

The present invention provides recombinant fusion (chimeric) proteins comprising transferrin binding proteins and a mucosal adjuvant, and their use to broadly elicit an immune response to different strains of a Neisserial species, such as N. gonorrhoeae or N. meningitidis. Significantly, when administered intranasally, such fusion proteins elicit antibody production in mucosal secretions, for example, in the urogenital or oropharynx tracts. This is important because Neisseria infections are typically contracted when the bacteria invade mucosal surfaces. In some embodiments, the fusion protein comprises one or more Neisseria transferrin binding proteins (or antigenic regions thereof) and a mucosal adjuvant such as a toxin molecule, or portion or subunit of a toxin molecule, such as the B-subunit of cholera toxin or Escherichia coli heat labile toxin II. For example, the fusion proteins may comprise transferrin binding proteins A and/or B of Neisseria species N. gonorrhoeae or N. meningitidis, or antigenic regions of those proteins (e.g. L2 or NB domains), fused to the B-subunit of cholera toxin or Escherichia coli heat labile toxin type II. Significantly, the antibodies that are elicited by such fusion proteins are cross-bactericidal with several heterologous bacterial strains within the Neisseria species from which the transferrin binding protein originates.

It is an object of this invention to provide a fusion protein comprising one or more transferrin binding proteins or antigenic regions thereof, and a mucosal adjuvant. In some embodiments, the transferrin binding protein is a Neisseria transferrin binding protein such as ThpA or TbpB, which may originate from, for example, Neisseria gonorrhoeae or Neisseria meningitidis. In one embodiment, the antigenic region is L2 or NB. In some embodiments of the invention, the mucosal adjuvant originates from a toxin molecule, for example, from Vibrio cholerae or Escherichia coli. In some embodiments, the mucosal adjuvant is either the B subunit of Vibrio cholerae toxin or the B subunit of Escherichia coli heat labile toxin type II.

The invention also provides a method of eliciting antibodies to one or more transferrin binding proteins, or antigenic regions thereof, in a mammal. The method includes the step of administering to the mammal a fusion protein comprising one or more transferrin binding proteins or antigenic regions thereof and a mucosal adjuvant in an amount sufficient to elicit antibodies to a transferrin binding protein in the mammal. In one embodiment, the step of administering is carried out intranasally. In yet another embodiment of the invention, antibodies are produced in one or more locations in the mammal, the locations being, for example, the urogenital tract, the oropharynx tract or serum. The antibodies may be of class IgA. Alternatively (or in addition), the antibodies may include bactericidal IgG antibodies. In some embodiments, the fusion protein comprises a transferrin binding protein or antigenic region thereof, and a mucosal adjuvant. In some embodiments, the transferrin binding protein is a Neisseria transferrin binding protein such as ThpA or TbpB, which may originate from, for example, Neisseria gonorrhoeae or Neisseria meningitidis. In one embodiment, the antigenic region is L2 or NB. In some embodiments of the invention, the mucosal adjuvant originates from a toxin molecule, for example, from Vibrio cholerae toxin or Escherichia coli heat labile toxin II. In some embodiments, the mucosal adjuvant is either the B subunit of Vibrio cholerae toxin or the B subunit of Escherichia coli heat labile toxin II.

The invention further provides an amino acid sequence as represented by SEQ ID NO: 10, and nucleotide sequences that encode the amino acid sequence represented by SEQ ID NO: 10.

The invention further provides a nucleotide sequence encoding one or more Neisseia transferrin binding proteins and at least one mucosal adjuvant.

The invention further provides a method for inhibiting growth of Neisseria on a mucosal surface of a patient in need thereof. The method comprises the step of administering to the patient a fusion protein or polypeptide, or antibodies to the fusion protein or fusion polypeptide. The fusion protein or polypeptide comprises one or more Neisseria transferrin binding proteins or antigenic regions thereof, and a mucosal adjuvant. The one or more Neisseria transferrin binding proteins may be, for example, transferrin binding protein A or transferrin binding protein B, or both. The one or more antigenic regions may be, for example, L2 or NB, or both.

### BRIEF DESCRIPTION OF THE DRAWINGS

FIG. 1A-C. A, Amino acid sequence of transferrin binding protein A of N. gonorrhoeae (SEQ ID NO: 1) from strain FA19; B-C, nucleic acid sequence encoding transferrin binding protein A of N. gonorrhoeae (SEQ ID NO: 2) from strain FA19.

FIG. 2A-D. A, Amino acid sequence of transferrin binding protein B of N. gonorrhoeae (SEQ ID NO: 3) from strain FA19; B-D, nucleic acid sequence encoding transferrin binding protein B of N. gonorrhoeae (SEQ ID NO: 4) from strain FA19.

FIG. 3. Amino acid sequence of mature transferrin binding protein B of N. gonorrhoeae (SEQ ID NO: 5) with amino terminal cysteine removed.

FIG. 4 A-C. Amino acid sequences of NB, L2 and L2/NB fusion. A. NB (SEQ ID NO: 6); B, L2 (SEQ ID NO: 7); C, L2/NB fusion (SEQ ID NO: 8). Underlined sequence “LEGS” is derived from XhoI and BanHI restriction sites.

FIG. 5. Schematic representation of generic ThpA showing loops L1-L11 and ThpA-based peptides ThpA-2 to ThpA-8.

FIG. 6. Multisequence alignment for TbpB from several strains of N. gonorrhoeae and N. meningitidis. “g” indicates an N. gonorrhoeae strain; “m” indicates an N. meningitidis strain. Highly conserved regions 1-6 are shown as cross-hatched bars under the sequence.

FIG. 7. Growth inhibition of strains FA19 and FA1090 grown in the presence of day 63 vaginal wash antibodies and hTf as a sole iron source. Panel A demonstrates growth inhibition of strain FA19 in the presence of either NB-Ctb or NB-L2-Ctb vaginal wash samples diluted 1/10. Panel B demonstrates growth inhibition of strain FA1090 in the presence of NB-L2-Ctb vaginal wash samples diluted 1/10. Controls were pooled vaginal wash samples from sham immunized mice obtained on day 63. Each growth condition was performed in duplicate, with optical density levels measured every 2 hours. Graphs are representative of 3 separate experiments.

FIGS. 8A and B. Sequence alignments of the NB and L2 domains. A, amino acid sequence alignment of the NB domain of TbpB from strains FA19 (SEQ ID NO: 39), MS11 (SEQ ID NO: 40, the encoding region of which has GenBank Accession #EF547129), and FA1090 (SEQ ID NO: 41). B, amino acid sequence alignment of the L2 domain of ThpA from FA19 (SEQ ID NO: 42), MS11 (SEQ ID NO: 43, the encoding region of which has GenBank Accession #EF547130), and FA1090 (SEQ ID NO: 44). The MS11 sequences, determined as part of this study, lack the terminal residues of NB and L2 domains since the regions encoding these amino acids were amplified with FA19-specific primers.

### DETAILED DESCRIPTION OF THE PREFERRED EMBODIMENTS OF THE INVENTION

The present invention provides novel recombinant fusion (chimeric) proteins comprising transferrin binding proteins and a mucosal adjuvant. It has been discovered that such fusion proteins, when administered to a mammal, elicit an immune response to the transferrin binding proteins in the mucosal secretions of the mammal. In particular, when such fusion proteins are administered intranasally to a host, antibodies are produced in mucosal secretions of the urogenital and/or oropharynx tracts. The ability to elicit an immune response in the mucosal secretions of a mammal is important because Neisseria infections are typically contracted through invasion of mucosal surfaces. In some embodiments of the invention, the fusion protein comprises Neisseria transferrin binding proteins A and/or B of N. gonorrhoeae or N. meningitidis, or antigenic portions of those proteins. A second component of the fusion protein is a mucosal adjuvant. The mucosal adjuvant portion may be, for example, a toxin molecule or a portion or subunit of a toxin molecule. For example, in some embodiments of the invention, the transferrin binding proteins are fused to the B-subunit of cholera toxin or Escherichia coli heat labile toxin (LTIIb). Importantly, antibodies elicited by administration of the fusion proteins are cross-bactericidal against heterologous bacterial strains, i.e. administration of a single fusion protein causes the recipient to mount a broad immune response against several immunologically related strains of the species of Neisseria from which the transferrin binding protein originates.

The neisserial transferrin binding proteins and antigenic regions thereof that are utilized in the invention are recombinant transferrin binding proteins, or antigenic regions thereof. The term “recombinant” as used herein should be understood to have the meaning that is well-recognized in the art. Typically, a protein or nucleic acid sequence of interest is identified in a natural source such as a bacterium. The bacterium is thus the source from which the transferrin binding protein originates, i.e. the protein sequence “originates from” or is “based on” the sequence from the bacterium, it being realized that such sequences can be produced by a variety of different mechanisms such as by recombinant techniques or chemically. The nucleic acid sequence encoding the protein of interest is then cloned, purified and/or otherwise manipulated by molecular biology techniques to generate a recombinant nucleic acid sequence. The protein encoded by the recombinant nucleic acid sequence may be expressed as a recombinant protein in any of several methods that are known to those of skill in the art.

The recombinant transferrin binding proteins that are utilized in the present invention are at least partially surface exposed proteins or antigenic regions of proteins from Neisseria species. The recombinant transferrin binding proteins demonstrate sufficient antigenicity to elicit an immune response in a mammal to which they are administered, and the immune response produces antibodies specific to one or more strains of the Neisseria bacterium from which the protein originates. The antibodies produced are capable of binding to the Neisseria bacteria, and in one embodiment, are bactericidal, i.e. they are IgG antibodies and kill the bacteria in the presence of human complement components. However, in some embodiments, the bacteria may not be killed outright but may be otherwise prevented from causing disease symptoms typically associated with infection by the bacteria, or such disease symptoms may occur but in a milder form. For example, the antibodies may thwart the ability of the bacterium to enter the cell, to reproduce, etc.

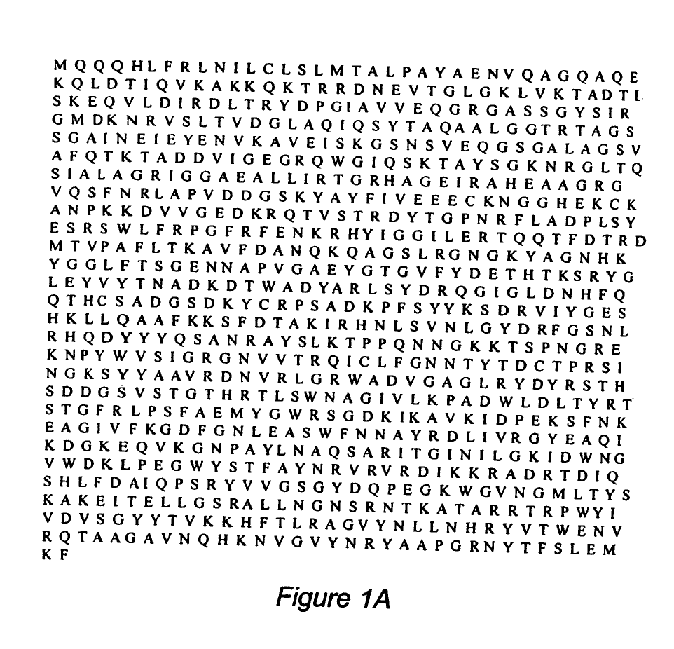

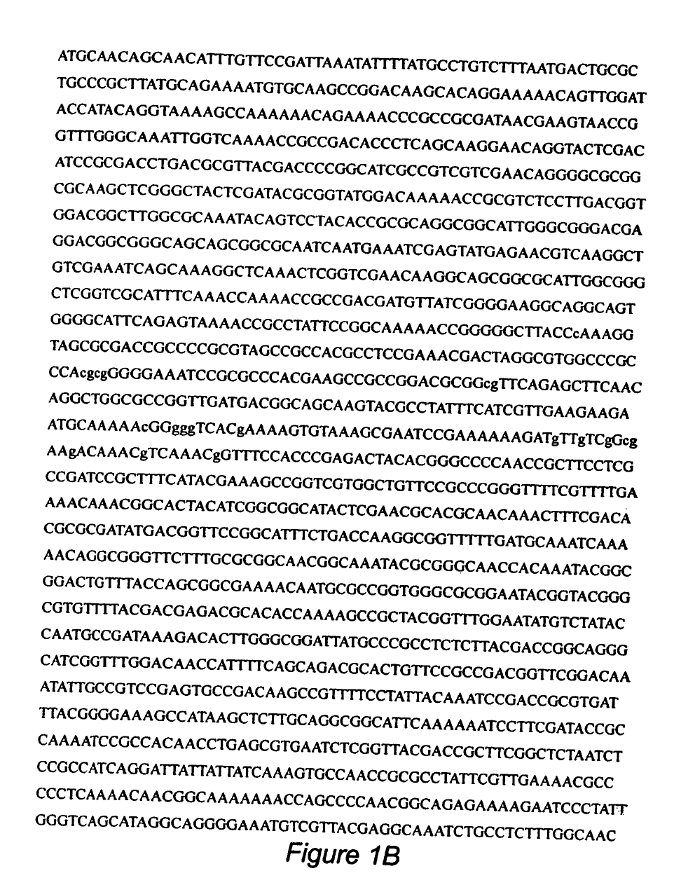

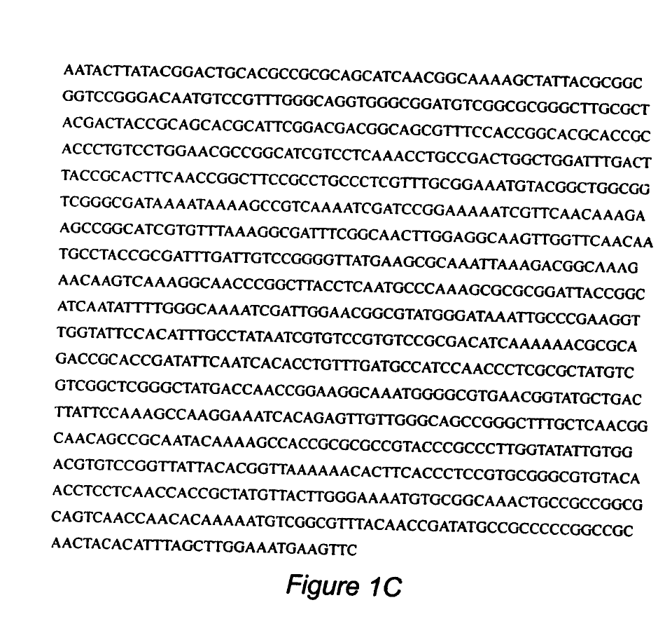

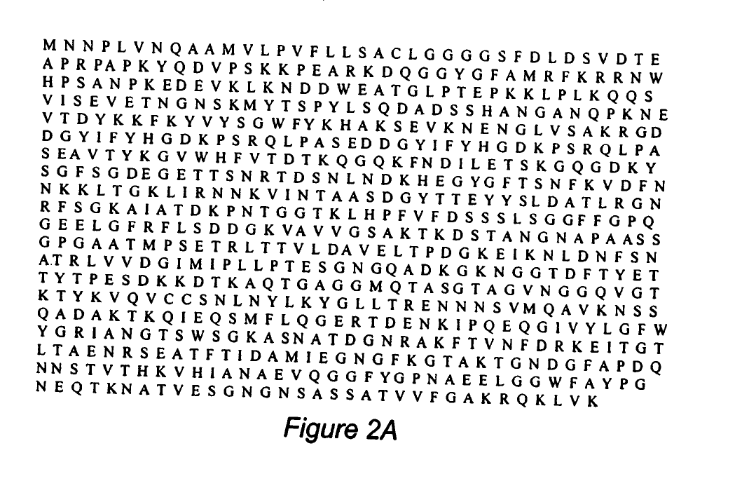

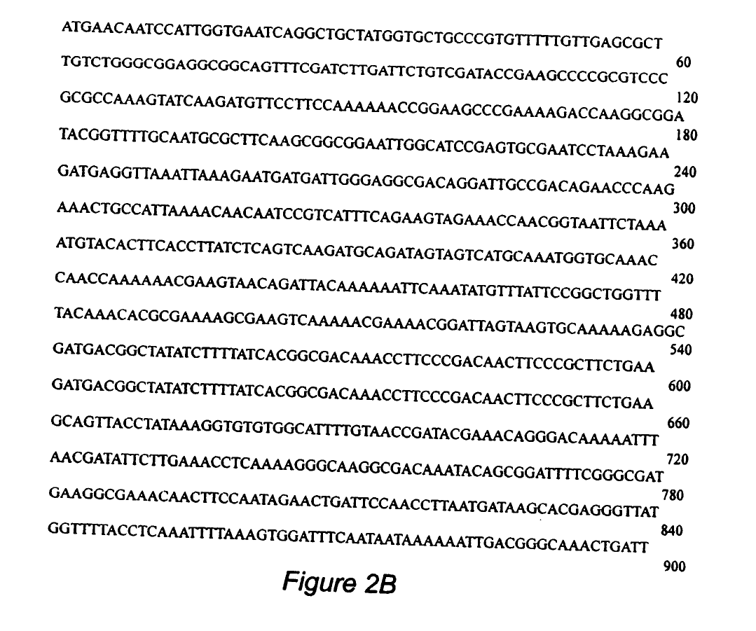

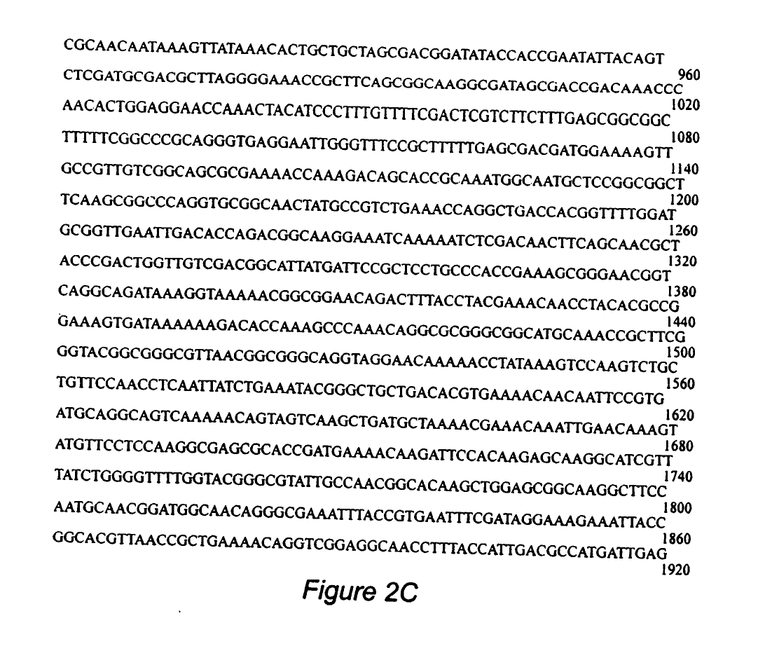

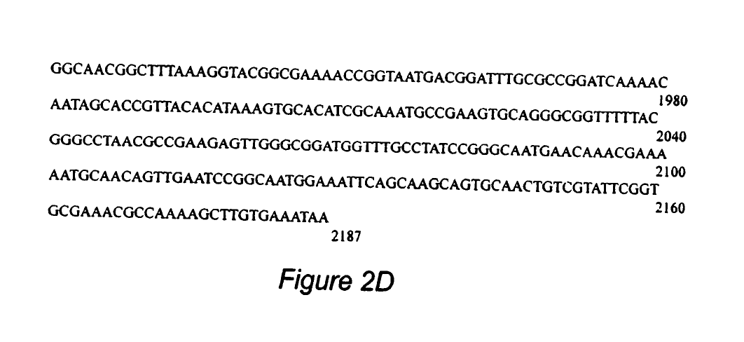

In some embodiments of the invention, the transferrin binding proteins are transferrin binding proteins A and B (ThpA and TbpB, respectively) from either Neisseria gonorrhoeae or Neisseria meningitidis. FIG. 1A-C and FIG. 2A-D show exemplary amino acid primary sequences and the nucleic acid sequences encoding N. gonorrhoeae ThpA (SEQ ID NO: 1, amino acid; SEQ ID NO:2, nucleic acid) and TbpB (SEQ ID NO: 3, amino acid; SEQ ID NO: 4, nucleic acid), respectively from strain FA19. Those of skill in the art will recognize and have access to other sequences encoding N. gonorrhoeae ThpA and N. gonorrhoeae TbpB, for example, those from other strains of the organism, which can also be used in the practice of the invention. For example: ThpA from strain FA1090 (GenBank Accession number AF124339); strain UU1008 (GenBank Accession number AF124338) strain Pgh3-2 (GenBank Accession number AF241227); and strain 4102 (GenBank Accession number AF240638); and TbpB from strain FA1090 (GenBank Accession number U65219); strain UU1008 (GenBank Accession number U65222) and strain Pgh3-2 (GenBank Accession number U6i5221).

Likewise, for N. meningitidis ThpA and TbpB, those of skill in the art will recognize and have access to several suitable sequences, for example: for ThpA, strain M982 (a high molecular weight class, GenBank Accession number Z15130) and strain B16B6 (a low molecular weight class, GenBank Accession number Z15129); and for TbpB, strains M982 (a high molecular weight class, GenBank Accession number Q09057) and strain B16B6 (a low molecular weight class, GenBank Accession number Q06988).

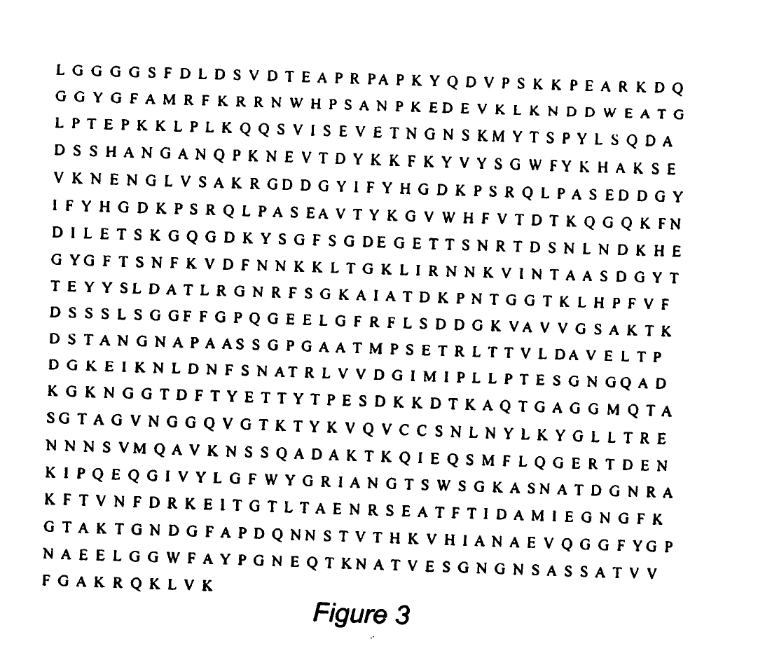

While the fission proteins may include such proteins in entirety, portions of the proteins may also be utilized. For example, as described in the Examples sections below, fusion proteins may be constructed with portions of ThpA and TbpB such as TbpB which is based on the mature protein (SEQ ID NO: 5, FIG. 3) but lacks the amino terminal cysteine residue. This is illustrated in FIG. 3, where the amino terminal cysteine of the mature protein is shown in brackets.

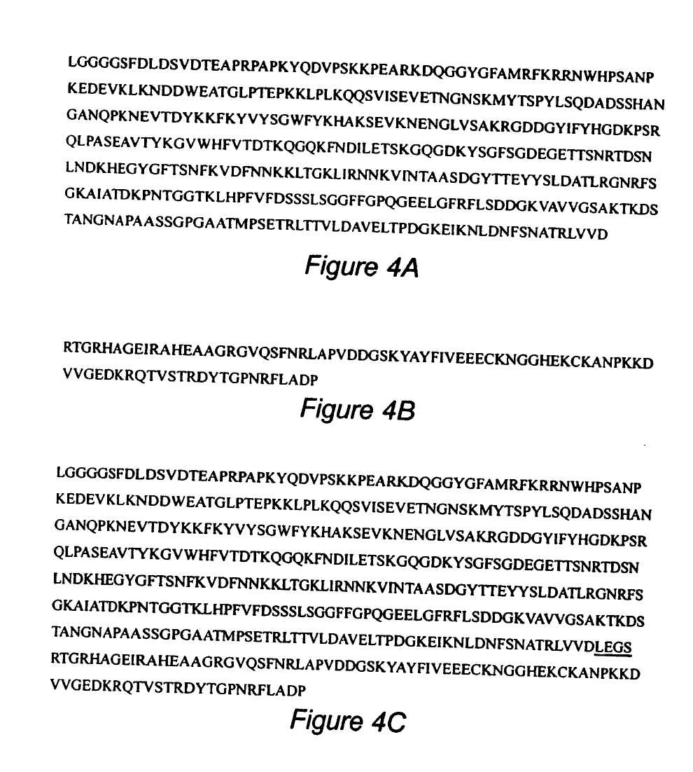

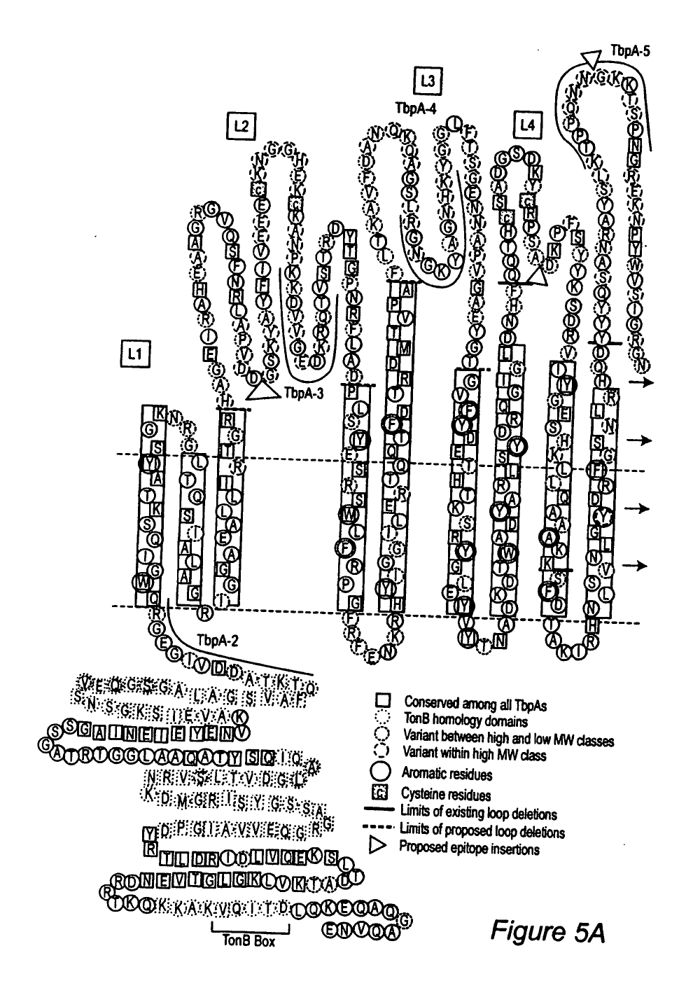

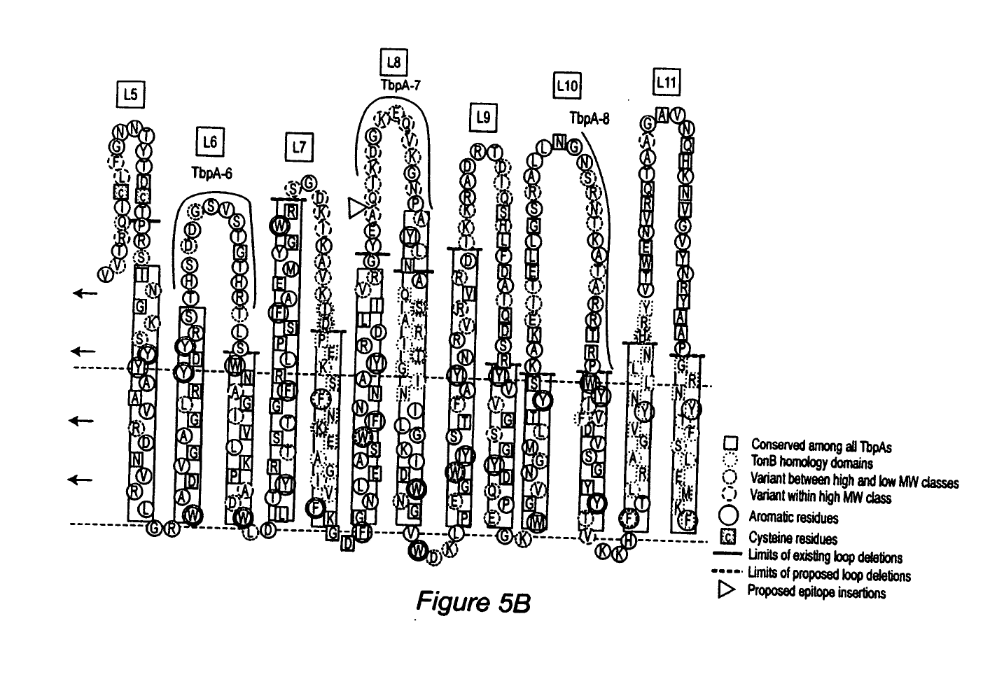

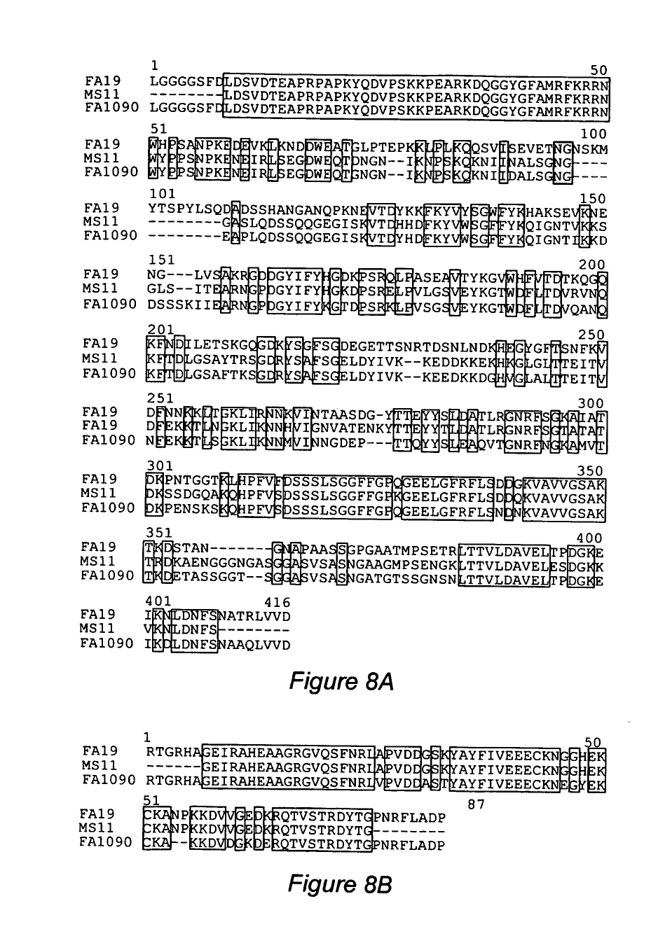

Further, antigenic regions of the Tbp proteins may also be used in the practice of the invention. By “antigenic region” we mean a portion of the full-length amino acid sequence of the protein that, by itself, elicits an immune response in a mammal to which it is administered. Such antigenic regions will generally be from about 5 to about 100 or more amino acids in length, or preferably about 10 to about 90, or about 10 to about 80, or about 10 to about 70, or about 10 to about 60, or about 10 to about 50, or about 10 to about 40, or about 10 to about 30, or even from about 10 to about 20 amino acids in length. For example, the L2 and NB regions of ThpA and TbpB, respectively, are antigenic regions of ThpA and TbpB. The amino acid sequence of L2 from strain FA19 (SEQ ID NO: 6) is provided in FIG. 4B, and all nucleotide sequences encoding this amino acid sequence are also encompassed by this invention. The amino acid sequence of NB from strain FA19 (SEQ ID NO: 8) is provided in FIG. 4A, and all nucleotide sequences encoding this amino acid sequence are also encompassed by this invention. Other possible antigenic regions for ThpA are illustrated in FIG. 5, which shows a diagram of ThpA indicating surface exposed loops, sequences which span the membrane, and sequences which are largely not surface exposed. Antigenic regions illustrated in this figure include loops L1-L11, and selected peptides ThpA-2-ThpA-8, all of which are encompassed by the phrase “antigenic regions” as used herein. Additional information concerning TbpA-2-ThpA-8 is also provided in Cornelissen et al., 2000, Inf. and Immun. 68: 4725-4735. The amino acid sequences of ThpA 2-ThpA-8 are given in Table 1. For TbpB, the amino acid sequences of exemplary antigenic regions are also given in Table 1 as TbpB-1-TbpB-9. Further exemplary NB and L2 sequences from strain MS11 are shown in FIGS. 8A and 8B, respectively, which also shows the alignment of NB and L2 from FA19, MS11 and FA1090, all of which sequences are encompassed by the present invention, as are nucleic acid sequences (DNA, RNA, etc.) that encode them.

TABLE 1

Antigenic peptides of TbpA and TbpB from
strain FA19 strain

| | | | | | |
|---|---|---|---|---|---|
| | | Length | | | |
| | | (amino | ID | MC | ID MC |
| Name | Sequence | acids) | GC^{a} | (low)^{b} | (high)^{c} |

| | | | | | |
|---|---|---|---|---|---|
| TbpA | | | | | |
| Tbp-A-2 | QTKYADDVIGEGRQ | 14 | 100% | 100% | 100% |
| | (SEQ ID NO: 19) | | | | |
| Tbp-A-3 | KKDVVGEDKRQT | 12 | 75% | 50% | 100% |
| | (SEQ ID NO: 20) | | | | |
| Tbp-A-4 | RGNGKYAGNHK | 11 | 64% | 55% | 64% |
| | (SEQ ID NO: 21) | | | | |
| Tbp-A-5 | KTPPQNNGKKTSPN | 14 | 86% | 36% | 50% |
| | (SEQ ID NO: 22) | | | | |
| Tbp-A-6 | HSDDGSVSTGTHR | 133 | 100% | 85% | 100% |
| | (SEQ ID NO: 23) | | | | |
| Tbp-A-7 | KDGKEQVKGNP | 11 | 73% | 27% | 73% |
| | (SEQ ID NO: 24) | | | | |
| Tbp-A-8 | NSRNTKATARRTRP | 14 | 100% | 57% | 100% |
| | (SEQ ID NO: 25) | | | | |
| TbpB | | | | | |
| Tbp-B-1 | PSKKPEARKDQ | 11 | 81% | 27% | 64% |
| | (SEQ ID NO: 26) | | | | |
| Tbp-B-2 | NQPKNEVTYKK | 12 | 8% | 17% | 50% |
| | (SEQ ID NO: 27) | | | | |
| Tbp-B-3 | TDTKQGQKFND | 11 | 45% | 36% | 64% |
| | (SEQ ID NO: 28) | | | | |
| Tbp-B-4 | DEGETTSNRTDSNLND | 19 | 5% | 16% | 47% |
| | KHE | | | | |
| | (SEQ ID NO: 29) | | | | |
| Tbp-B-5 | DFNNKKLTGKLIRNNK | 16 | 56% | 50% | 87% |
| | (SEQ ID NO: 30) | | | | |
| Tbp-B-6 | TPDEKEIKNLDNFSNA | 18 | 61% | 18% | 75% |
| | TR | | | | |
| | (SEQ ID NO: 31) | | | | |
| Tbp-B-7 | TYETTYTPESDKKDTK | 18 | 47% | NP^{d} | 58% |
| | AQT | | | | |
| | (SEQ ID NO: 32) | | | | |
| Tbp-B-8 | KNSSQADAKTKQ | 12 | 25% | NP | 83% |
| | (SEQ ID NO: 33) | | | | |
| Tbp-B-9 | QGERTDENKIPQEQ | 14 | 64% | 36% | 71% |
| | (SEQ ID NO: 34) | | | | |

^{a}Percent identity scores were calculated from multiple sequence alignment, comparing 5 gonococcal sequences.
^{b}Percent identity scores were calculated from pairwise sequence alignment, comparing FA19 Tbp sequence with meningococcal Tbp sequence from low molecular weight class (B16B6)
^{c}Percent identity scores were calculated from pairwise sequence alignment, comparing FA19 Tbp sequence with meningococcal Tbp sequence from high molecular weight class (M982)
^{d}sequence is not present in meningococcal strain B16B6

Thus, the present invention also comprehends recombinant proteins comprising at least a portion of one or more antigenic region. Further, such antigenic regions may be incorporated into fusion proteins (e.g. with a mucosal adjuvant) either alone, or in combination with other antigenic regions. One such exemplary fusion protein includes both NB and L2, which has been genetically engineered to be translated within a single contiguous polypeptide. The amino acid sequence of the resulting polypeptide (SEQ ID NO: 8) is presented in FIG. 4C. All nucleotide sequences that encode this sequence are also encompassed by the present invention. In fusion proteins containing multiple, recombinant antigenic regions, the antigenic regions may be encoded and translated in tandem, or may be separated by spacers between the antigenic regions in the encoding nucleic acid sequence, or in the polypeptide sequence, or both. Further, such fusion proteins may include one or more different antigenic regions, and/or one or more copies of a single antigenic region.

The invention also encompasses variant recombinant fusion proteins comprising amino acid sequences that are derived from the sequences disclosed herein, for example, the sequences presented as SEQ ID NOS: 1, 3, or 5, or selected regions thereof. By “derived from” we mean that the sequence displays at least about 50 to 100% identity to the amino acid sequences disclosed herein, or about 60 to 100% identify, or about 70 to 100% identity, or even from about 70 to 100% or about 80 to 100% identify. In preferred embodiments, the variant sequences display from about 90 to 100% or about 95 to 100% amino acid identity. Variations in the sequences may be due to a number of factors, for example, conservative or non-conservative amino acid substitutions, natural variations among different strains or species, deletions or insertions, addition of leader sequences to promote secretion from the cell, etc. Such alterations may be naturally occurring or may be purposefully introduced for any of a wide variety of reasons, e.g. in order to eliminate or introduce protease cleavage sites, to eliminate or introduce glycosylation sites, in order to improve solubility of the protein, to facilitate protein isolation (e.g. introduction of a histidine tag), as a result of a purposeful change in the nucleic acid sequence (see discussion of the nucleic acid sequence below) which results in a change in one or more codons and thus the translated amino acid, etc. All such variant sequences are encompassed by the present invention, so long as the resulting protein (polypeptide or peptide) functions in the fusion protein to elicit production of antibodies in the mammal to which the fusion protein is administered. For example, it is noted that the mature TbpB protein lacks the first twenty amino acids depicted in FIG. 2A, and thus the amino terminal residue of the mature protein in vivo is cysteine. The present invention also encompasses the use of such a protein, i.e. from amino acids 21 (cysteine) to the carboxy terminal lysine, and variants thereof, as described herein.

Further, the invention also comprehends the use of protein variants that are shorter than SEQ ID NOS: 1 and 3, so long as the shorter protein functions in the fusion protein to elicit production of antibodies in the mammal to which the fusion protein is administered. For example, SEQ ID NO: 5 (FIG. 3) lacks the leader sequence and amino terminal cysteine residue of the SEQ ID NO: 3. Other amino and carboxyl terminal deletion variants may also be designed, as well as other variants from which internal sequences have been deleted. All such variant sequences are encompassed by the present invention, so long as the resulting protein functions in the fusion protein to elicit production of antibodies in a mammal to which the fusion protein is administered, preferably in the mucosa of the mammal. In addition, the fusion proteins of the invention may comprise only one Thp or variant thereof, or may contain multiple copies of one Thp or variant, or may contain copies of several different Tbps or variants thereof.

A second moiety in the fusion proteins of the invention is a mucosal adjuvant. By “mucosal adjuvant” we mean an entity that elicits an immune response in the mucosa. Those of skill in the art will recognize that such an adjuvant may also elicit a systemic immune response as well, and in a preferred embodiment, both a systemic and mucosal immune response is elicited. In one embodiment of the invention, the mucosal adjuvant is a toxin molecule that has been manipulated to attenuate or remove its toxicity. For example, a non-toxic subunit of a toxin molecule may be utilized. Those of skill it the art will recognize that several such molecules exist, including but not limited to molecules which originate with Vibrio cholerae or E. coli, such as the pentavalent B-subunits of cholera toxin and E. coli heat labile toxin II. Any mucosal adjuvant may be utilized in the practice of the invention, so long as the resulting protein functions in the fusion protein to elicit production of bactericidal antibodies in the mucosa of a mammal to which the fusion protein is administered intranasally.

The invention also comprehends nucleic acid sequences that encode the fusion proteins of the invention. Such nucleic acid sequences encode both the transferrin binding protein moiety and the mucosal adjuvant moiety. With respect to the transferrin binding protein moiety, exemplary nucleic acid sequences are represented by SEQ ID NOS: 2 and 4, which encode the proteins represented by SEQ ID NOS: 1 and 3, respectively. However, as is well known, due to the degeneracy of the nucleic acid triplet code, many other nucleic acid sequences that would encode the same protein sequences could also be designed, and the invention also encompasses such nucleic acid sequences. Further, as described above, many useful variant forms of the proteins of the invention also exist, and nucleic acid sequences encoding such variants are intended to be encompassed by the present invention. Further, such nucleic acid sequences may be varied for any of a variety of reasons, for example, to facilitate cloning of the moieties of the fusion protein, to facilitate transfer of the fusion protein clone from one construct to another, to add or replace promoter sequences, etc. In addition, all genera of nucleic acids (e.g. DNA, RNA, various composite and hybrid nucleic acids, etc.) corresponding to the fusion proteins of the invention are intended to be encompassed by the invention.

The invention further comprehends vectors which contain nucleic acid sequences encoding the fusion proteins of the invention. Those of skill in the art are familiar with the many types of vectors which can be useful for such a purpose, for example: plasmids, cosmids, various expression vectors, viral vectors, etc.

Production of the fusion proteins of the invention can be accomplished in any of many ways that are known to those of skill in the art. For example, the protein may be made from a plasmid contained within a bacterium such as E. coli, in an insect expression system, in a yeast expression system, plant cell expression systems, etc. To that end, the present invention also encompasses a host cell that has been transformed or otherwise manipulated to contain nucleic acids encoding the fusion proteins of the invention, either as extra-chromosomal elements, or incorporated into the chromosome of the host.

By “elicit an immune response” we mean that, when administered to a mammal, the fusion proteins of the invention cause the host to mount an immune response to the protein. In other words, antibodies against the protein are generated. The amount of antibody that is generated may vary from fusion protein to fusion protein, or from mammal to mammal, but the titer will generally be in the range of about 0.1 μg/mL to about 10 μg/mL or more, and preferably in the range of about 0.5 μg/mL to about 10 μg/mL or more, with responses in the range of about 1 μg/mL to about 10 μg/mL or more being considered as “highly antigenic”.

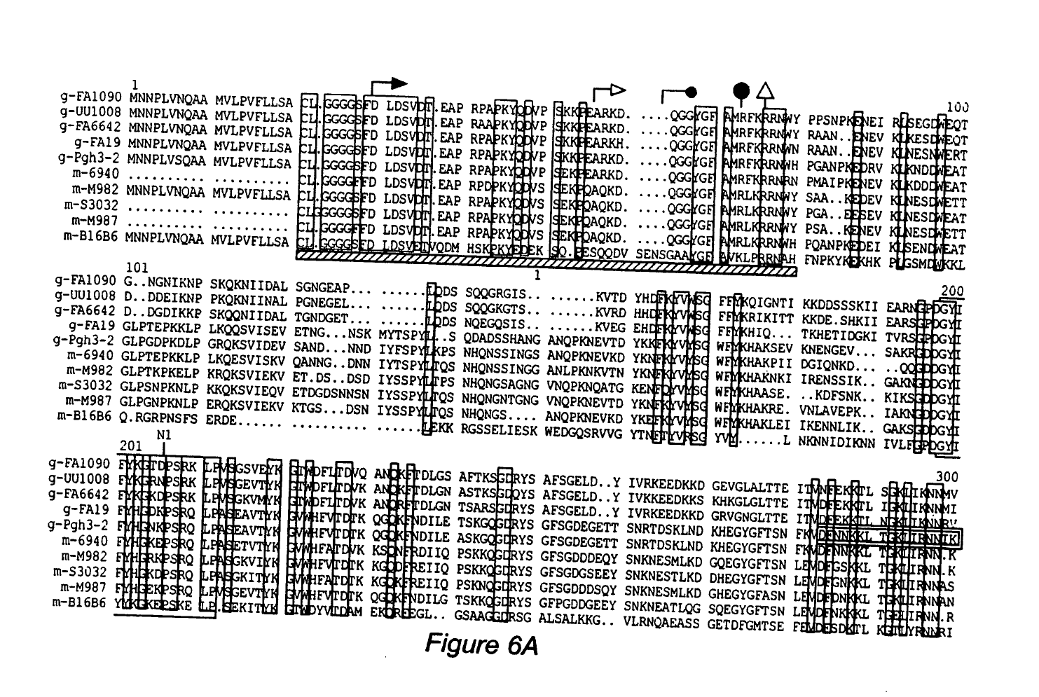

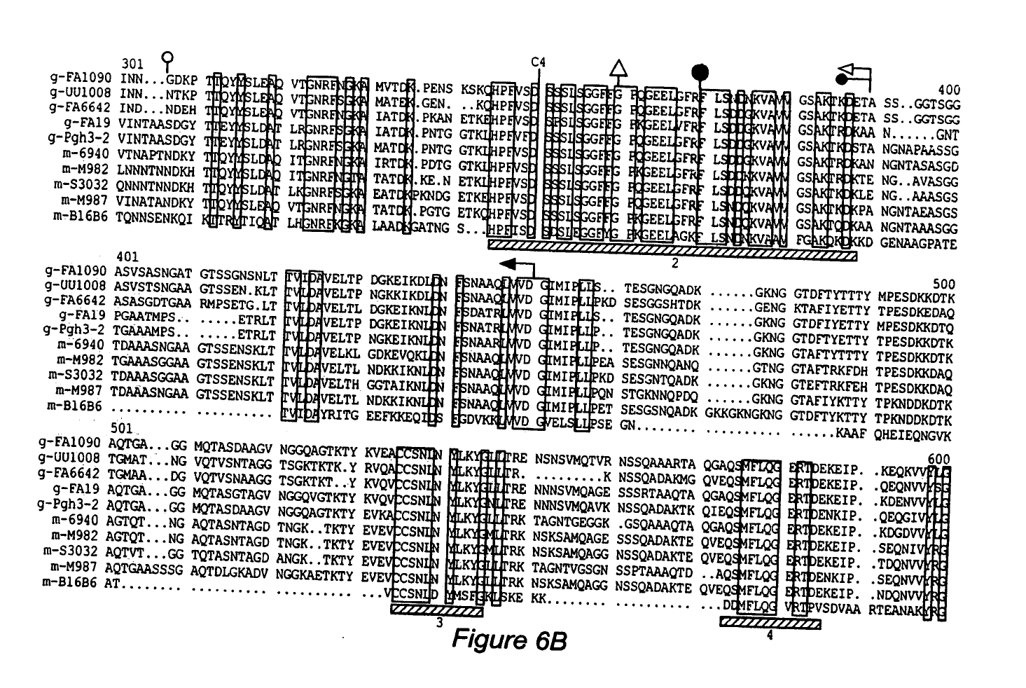

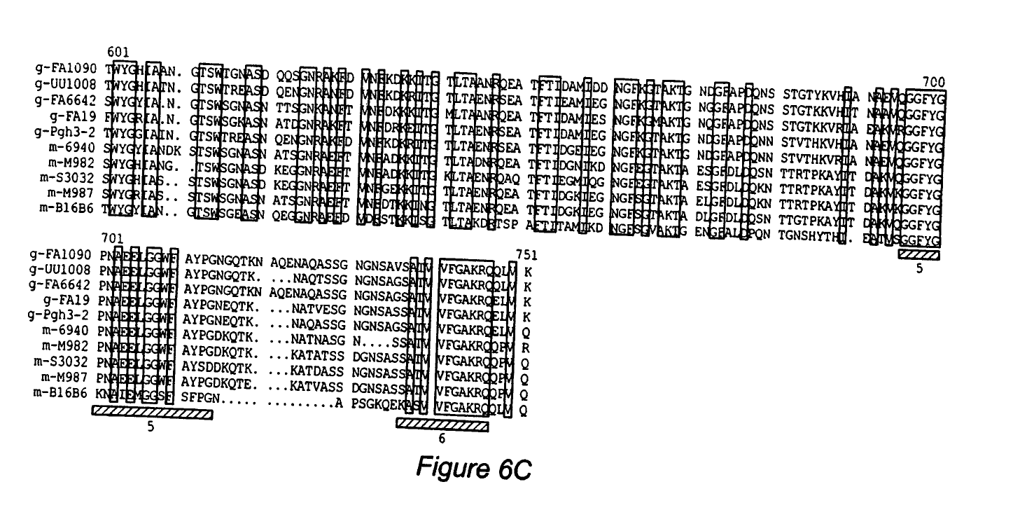

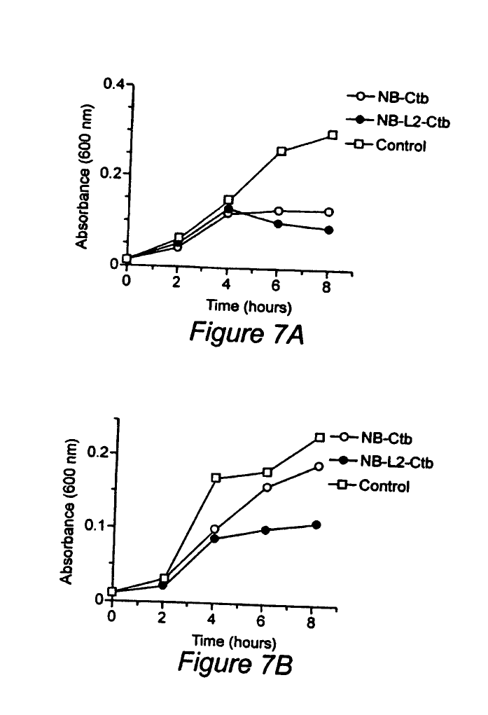

It should be noted, however, that the present invention is also based on the understanding that the most significant characteristic of an antibody that is elicited by the fusion proteins of the invention is not necessarily the amount of antibody that is made, but rather the type of antibody, the structural or functional significance of the epitope to which the antibody is directed, and the ability of the antibody to cross-react with related strains or species. In other words, counter to conventional approaches to vaccine development, the present invention is in part based on the discovery that even epitopes or regions that do not elicit a high antibody titer (and would not typically be considered “highly antigenic”) can still cause the production of efficacious antibodies that are bactericidal and protective against challenge with Neisserial species. For example, peptides representing highly conserved regions of Tbps may be used in the practice of the present invention. Such regions are not necessarily surface exposed, and/or may be only partly or intermittently exposed at the surface of the protein. Nevertheless, due to their important (or even critical) function in the protein (which may account for their high conservation across strains and species), antibodies against these regions tend to be highly efficacious. Those of skill in the art are well-acquainted with methods for identifying such highly conserved regions, and for determining the location of such regions with respect to the protein sequence. By “highly conserved” we mean that a primary amino acid sequence displays an identity of at least about 50 to 100%, and preferably about 60 to 100% and more preferably about 70 to 100%, and most preferably about 80 to 100% or even 90 or 95 to 100% identity, when compared to the corresponding aligned primary amino acid sequence of proteins from other strains of the same bacterial species. Those of skill in the art are well-acquainted with methodology for aligning primary sequences from different proteins, polypeptides or peptides. For example, FIG. 6 depicts a multisequence alignment of TbpB sequences from a variety of N. gonorrhoeae and N. meningitidis strains in which highly conserved amino acids are shown boxed in black, and highly conserved regions 1-6 are indicated by a numbered hatched bar under the sequence. In FIG. 6, “g” indicates an N. gonorrhoeae strain, and the sequences presented are from strain FA1090 (SEQ ID NO: 9), strain UU1008 (SEQ ID NO: 10); strain 10 FA6642 (SEQ ID NO: 11); strain FA19 (SEQ ID NO: 12); and strain Pgh3-2 (SEQ ID NO: 13). In FIG. 6, “n” indicates an N. meningitidis strain, and the sequences presented are from strain 6940 (SEQ ID NO: 14); strain M982 (SEQ ID NO: 15); strain 93032 (SEQ ID NO: 16); strain M987 (SEQ ID NO: 17); and strain B16B6 (SEQ ID NO: 18).

In addition, the “plug” region of ThpA (indicated by brackets in FIG. 5) while believed to be located largely within the membrane, is also highly conserved and may provide antigenic regions for use in the invention. Regions such as those illustrated, while being highly conserved but not necessarily “highly antigenic”, are still capable of eliciting antibodies of interest when utilized in the methods of the invention, and are intended to be encompassed by the phrase “antigenic regions”. For example, one or more amino acid sequences based on such conserved regions may be included in the fusion proteins of the invention. By “based on” we mean “derived from” with a meaning as described above, or including such regions, or displaying at least about 75 to 100% identity to such regions, or preferably about 80 to 100%, or more preferably about 85 to 100%, and most preferably about 90 to 100% or even about 95-100% identity with such regions. Such a fusion protein may contain one or more of such amino acid sequences from the same or different Tbp proteins from one or more strains or species. In addition, such fusion proteins may contain one or more amino acid sequences that are classified as “highly variable” or “highly antigenic”, e.g. epitopes in which the amino acid sequence varies from strain to strain or from species to species, and/or which typically elicit high antibody titers when administered to a mammal. A fusion protein may thus include amino acid sequences based on or derived from both highly conserved and highly variable regions of Thp proteins.

In a preferred embodiment, the fusion protein is administered intranasally and the antibodies are generated in the mucosa and serum of the mammal, particularly in the urogenital and/or oropharynx tracts, and especially in the vagina and urethra. Preferably, the antibodies will include IgA and IgG, although other classes of antibodies may also be produced. In addition, antibodies may be produced in serum. Serum production of antibodies is important to prevent dissemination of the organism.

The invention also provides antibodies that react with transferrin binding proteins. Such antibodies may be used, for example, for therapeutic purposes to treat or prevent disease symptoms associated with diseases caused by Neisseria species. Alternatively, such antibodies may be used for diagnostic purposes, e.g. to detect or identify Neisseria species, either in a clinical setting or in a laboratory setting. The antibodies may be of any known class, i.e. immunoglobulins IgA, IgG, IgM, IgD and IgE. Further, the antibodies may be either polyclonal or monoclonal, and may be produced in any manner known to those of skill in the art, including in host mammals such as rabbits or mice, or by molecular biology techniques.

The present invention further provides compositions for use in eliciting an immune response in a mammal. The compositions may be utilized as a vaccine against Neisseria species. In particular, the compositions elicit an immune response to Neisseria gonorrhoeae and/or Neisseria meningitidis. The compositions of the invention include a substantially purified transferrin fusion protein as described herein, and a pharmacologically suitable carrier. The preparation of such compositions for use as vaccines is well known to those of skill in the art. Typically, such compositions are prepared either as liquid solutions or suspensions, however solid forms such as tablets, pills, powders and the like are also contemplated. Solid forms suitable for solution in, or suspension in, liquids prior to administration may also be prepared. The preparation may also be emulsified. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. In addition, the composition may contain other adjuvants. If it is desired to administer an oral form of the composition, various thickeners, flavorings, diluents, emulsifiers, dispersing aids or binders and the like may be added. The composition of the present invention may contain any such additional ingredients so as to provide the composition in a form suitable for administration. The final amount of fusion protein antigen in the formulations may vary. However, in general, the amount in the formulations will be from about 1-99%. The compositions may further comprise an additional adjuvant, suitable examples of which include but are not limited to Seppic, Quil A, Alhydrogel, etc. The compositions may contain a single type of transferrin binding protein. Alternatively, more than one transferrin binding protein may be utilized in a preparation, i.e. the preparations may comprise a “cocktail” of such antigens.

In particular, in some embodiments of the invention, cocktails of antigens are used to more broadly protect against neisserial diseases. For example, chimeric proteins that include a cholera toxin B subunit (or other mucosal adjuvant) fused to portions of the Tbps are constructed from a representative Neisseria gonorrhoeae strain and from a representative Neisseria meningitidis strain. Characterized N. meningitidis strains fall into two broad classes, a “low molecular weight class” which expresses relatively smaller, and more divergent, Tbps (e.g. Strain B 16B6), and a “high molecular weight class”, which expresses larger Tbps (e.g. Strain M982). Protection against a plurality of strains is accomplished by including representatives from both classes in a single immunogenic preparation. The high molecular weight Tbps from meningococcal strains (and from M982 in particular) are very similar to those of all of the N. gonorrhoeae strains characterized to date. A mixture of chimeric proteins from N. meningitidis (for example, strain B16B6) and from N. gonorrhoeae (for example, strain FA19) can be combined into an immunogen cocktail, and the immune response generated is broadly protective against infection by most or all Neisserial isolates.

Alternatively, the compositions of the invention may include nucleic acids which encode the fusion proteins of the invention, i.e. the composition may be administered as a DNA vaccine.

The methods of the present invention involve administering a composition comprising one or more antigenic transferrin fusion protein in a pharmacologically acceptable carrier to a mammal. The vaccine preparations of the present invention may be administered by any of the many suitable means which are well known to those of skill in the art, including but not limited to by injection, orally, intranasally, by ingestion of a food product containing the antigen, etc. However, in preferred a embodiment, the mode of administration is intranasal. In addition, the compositions may be administered alone or in combination with other medicaments or immunogenic compositions, e.g. as part of a multi-component vaccine. Further, administration may be a single event, or multiple booster doses may be administered at various timed intervals to augment the immune response. In addition, administration may be prophylactic, i.e. before exposure to the bacteria has occurred, or is suspected to have occurred, or after the fact, i.e. after a known or suspected exposure, or therapeutically, e.g. after the occurrence of disease symptoms associated with bacterial infection.

### EXAMPLES

### Example 1

### Transferrin Binding Proteins Chemically Conjugated to Cholera Toxin Subunit B

The transferrin binding proteins (ThpA and TbpB) comprise the gonococcal transferrin receptor, and are considered potential antigens for inclusion in a vaccine against Neisseria gonorrhoeae. Intranasal immunization (IN) has shown promise in development of immunity against STD pathogens, in part due to the induction of antigen specific genital tract IgA and IgG. Conjugation of antigens to the highly immunogenic cholera toxin B subunit (Ctb) enhances antibody responses in the serum and mucosal secretions following IN vaccination. In the current study, we characterized the anti-Thp immune responses following immunization of mice IN with recombinant transferrin binding proteins (rThpA and rTbpB) conjugated to rctb. We found that both rThpA-Ctb and rTbpB-Ctb conjugates administered IN induced antibody responses in the serum and genital tract. IN immunization resulted in both IgA and IgG in the genital tract; however, subcutaneous immunization mainly generated IgG. Surprisingly, rThpA alone was immunogenic, and induced serum and mucosal antibody responses similar to those elicited against the rThpA-Ctb conjugate. Overall, rTbpB was much more immunogenic than rThpA, generating serum IgG levels that were greater than those elicited against rThpA. Bactericidal assays conducted with sera collected from mice immunized IN with TbpA and/or TbpB indicated that both antigens generated antibodies with bactericidal activity. Anti-ThpA antibodies were cross-bactericidal against heterologous gonococcal strains whereas TbpB-specific antibodies were less cross-reactive. By contrast, antibodies elicited via subcutaneous immunization were not cross-bactericidal against heterologous strains, indicating that IN vaccination could be the preferred route for elicitation of biologically functional antibodies.

### Materials and Methods

Construction of expression plasmids. The tbpA expression plasmid, pUNCH412, was described previously (13). The tbpB expression plasmid, pVCU711, was constructed by PCR amplification using a proofreading Taq polymerase (Platinum Pfx; Invitrogen) of a previously described tbpB expression plasmid, pVCU705 (34). The forward primer, oVCU240 (GGATCCTGTCTGGGCGGAGGCGGCAGTTTCG) (SEQ ID NO: 35), contained a BamHI site (shown in bold) and amplified the tbpB gene from the sequence that encodes amino acid 2 of the mature protein. The reverse primer, oVCU241 (CCCGGGTTATTTCACAAGCTTTTGGCGTTTCG) (SEQ ID NO: 36), contained a SmaI site (shown in bold) and encoded the stop codon of the FA19 tbpB gene. The PCR product was ligated into the pQE-80L expression vector (Qiagen). The resultant plasmid, pVCU711, encoded a recombinant TbpB in which the N-terminal six-histidine tag was fused to amino acid 2 of the mature protein. The resulting protein lacked the amino terminal cysteine residue and was expressed under the control of the T5 promoter. The ctb expression plasmid, pVCU710, was constructed by PCR amplification of the plasmid pCT^{ΔA1} (21). The forward primer, oVCU238 (TGGCCACACCTCAAAATATTACTGATTTGTGTG) (SEQ ID NO: 37) contained an MscI site (shown in bold) and amplified the mature ctb gene product. The reverse primer, oVCU239 (CTCGAGTTAATTTGCCATACTAATTGCGGCAATCG) (SEQ ID NO: 38), contained an XhoI site and amplified the 3′ end of the ctb gene, including the stop codon. The PCR product was ligated into the pET-22b(+) (Novagen) expression vector. The resultant plasmid, pVCU710, contained the mature ctb gene product fused with the E. coli pelB leader sequence immediately upstream. Gene expression was under the control of the T7 promoter. The expression host for pVCU710 and pVCU711 were the E. coli strains BL21(DE3) (Novagen), and TOP 10 (Invitrogen), respectively.

Recombinant protein expression and purif cation. Recombinant proteins were expressed in one-liter cultures of Luria-Bertani broth (LB) containing 1% glucose and 500 μg/ml of carbenicillin for rTbpA expression, or 200 μg/ml of ampicillin for rTbpB and rCtb expression. When the cultures reached an optical density at 600 nm of 0.4 to 0.6 they were induced with IPTG (isopropyl-B-D-thiogalactopyranoside). For rThpA, prior to induction, cultures were centrifuged for 15 min at 6000×g to pellet the bacteria. The pellets were then resuspended in fresh media as described above with 0.5 mM IPTG and allowed to express overnight at 27° C. (˜16 hrs). For rTbpB and rCtb expression, 0.5 mM IPTG was added and cultures allowed to express for 3 hours at 30° C. After induction, the cells were pelleted as described above and stored at −80° C.

For rThpA and rTbpB purification, pellets were thawed on ice and resuspended in Tris buffer (100 mM Tris (pH 8.0) and 0.5 M NaCl). After cells were completely resuspended, Elugent (Calbiochem) was added to a final concentration of 2%. Protease inhibitors (Sigma), lysozyme, and DNase were added and the mixture was allowed to incubate overnight at 4° C. Solubilized preparations were centrifuged at 18,000×g for 30 min. to remove insoluble material. ThpA was purified using a transferrin affinity column (26). The rThpA-transferrin column was washed with 20-bed volumes of 50 mM potassium phosphate (pH 8.0)-0.5M NaCl-0.05% Lauryl maltoside (n-dodecyl-B-D-maltopyranoside; Anatrace, Maumee, Ohio), and eluted with the above buffer at pH 2.0. The eluted proteins were immediately neutralized by the addition of 1 M potassium phosphate pH 8.0 and 0.05% lauryl maltoside. rTbpB was purified as described previously (34). Ctb pellets were resuspended in 50 mM potassium phosphate buffer pH 6.8 and 100 μg/mL lysozyme, and placed at 30° C. for 15 min. Following the 15 min. incubation, cell pellets were subjected to sonication on ice for 30 bursts repeated 3 times. Following centrifugation, supernatants were subjected to precipitation by ammonium sulfate, where Ctb precipitated at 60-80% saturation. The resulting precipitate was collected by centrifugation and dissolved in 20 mM potassium phosphate buffer, pH 6.8. The dissolved precipitate was dialyzed 3 times against a 1000-fold excess of potassium phosphate buffer. The dialyzed preparation was centrifuged to remove precipitated material, then passed through a 0.45-μM-pore-size syringe filter. Ctb was then purified by anion exchange chromatography using an Econo-Pac High S Cartridge (Biorad), and gel filtration using a Superdex 200 column (Amersham). Following purification, TbpB and Ctb were dialyzed 4 times against 1000-fold excess of PBS, and TbpA was dialyzed against PBS+0.05% lauryl maltoside.

Tbp-Ctb conjugate preparation. ThpA (1 mg in 1 mL PBS+0.05% Lauryl maltoside), TbpB (2 mg in 1 mL PBS), and Ctb (2 mg in 1 mL PBS) were treated with 5 μl of a 20 mM stock solution of SPDP (N-Succinimidyl 3-(2-pyridyldithio) propionate; Pierce) in DMSO for 1 hour at room temperature. Each protein was dialyzed against the corresponding initial buffers to remove free SPDP. To 1 mL of derivatized TbpA or TbpB, 0.5 mL of acetate buffer (100 mM sodium acetate, 100 mM NaCl, and 0.05% lauryl maltoside for ThpA only) containing 12 mg of dithiothreitol (DTT) was added and the mixture incubated for 30 min at room temperature. The reduced proteins were passed through a desalting column (Pierce), and protein concentrations were determined by BCA assay (Pierce). Equimolar amounts of derivatized Ctb was added to the reduced proteins and allowed to incubate overnight at 4° C. For ThpA conjugation, the derivatized Ctb was diluted to half by the addition of PBS+0.1% lauryl maltoside in order to keep the detergent concentration at 0.05%. Conjugated proteins were separated from unconjugated proteins by size exclusion chromatography using a Superdex 200 column (Amersham).

GM1 ganglioside ELISA. Purified conjugates were analyzed for the presence of Ctb and TbpA or TbpB using the GM1 ganglioside ELISA. ELISA plates (Nunc) were coated with 0.05 mL GM1 ganglioside (Sigma) diluted at 2?g/mL in methanol. Following evaporation of the methanol, the plates were blocked with 0.2 mL of PBS+1% skim milk for 1 hour at 37° C. The test samples were diluted at 1/100 in PBS or PBS+0.05% lauryl maltoside for the ThpA conjugate, and applied to each well in 0.1 mL volumes. The plate was then incubated at 30° C. for 1 hour. Plates were washed 3 times with PBS to remove unbound material, and bound conjugates were probed for 1 hour at room temperature with 0.05 mL of either anti-ThpA, anti-TbpB, or anti-CT (Sigma) rabbit sera diluted in PBS+1% skim milk. Plates were again washed as described above, and probed with 0.05 mL of alkaline phosphatase conjugated goat anti-rabbit IgG (Biorad) for 1 hour at room temperature. The plates were washed again and developed with 0.05 mL of p-nitrophenylphosphate substrate (Sigma) diluted in carbonate buffer (0.05 M sodium carbonate, 1 mM MgCl₂, pH 9.8). After sufficient color developed, the optical density of each well was measured at 405 μm and compared to blank and control wells.

Immunizations and sample collection. Female BALB/c mice, 7-8 weeks old were purchased from Harlan Sprague-Dawley (Indianapolis, Ind.). The mice were housed in microisolator cages and were under the care and supervision of the Division of Animal Resources. The protocols were approved by the Virginia Commonwealth University Institutional Animal Care and Use Committee. At the start of the experiment the mice were approximately 10 weeks old. Groups of five mice were immunized either intranasally or subcutaneously with Tbp-Ctb conjugate(s) or Tbps with or without Ctb as an adjuvant (see Table 2 for immunization details). All groups were immunized three times at 10 day intervals. Sera and vaginal secretions were collected on days 0, 17, 28, 35, and 65. Sera were obtained from tail vein blood samples and stored at −20° C. Vaginal secretions were obtained by pipeting 0.05 mL of PBS in and out of the vaginal vault 3 times. This procedure was repeated twice and the vaginal washes pooled. The protease inhibitor phenylmethylsulfonyl fluoride (Sigma) was added to each wash sample at a concentration of 1 mM following collection. Vaginal washes were kept at −80° C. until use.

TABLE 2
Immunization groups.

| Group | | |
|---|---|---|
| (Immunization Route) | Immunogen | Amount administered^{a} |
| A - Ctb (IN) | Ctb - TbpA conjugate | 20 mg |
| B - Ctb (IN) | Ctb - TbpB conjugate | 20 μg |
| A - Ctb + B - | Ctb - TbpA + Ctb - | 20 μg + 20 μg |
| Ctb (IN) | TbpB conjugates | |
| A + Ctb (IN) | Ctb + TbpA admixed | 10 μg + 10 μg |
| B + Ctb (IN) | Ctb + TbpB admixed | 10 μg + 10 μg |
| A + B + | Ctb + TbpA + TbpB | 10 μg + 10 μg + 10 μg |
| Ctb (IN) | admixed | |
| A only (IN) | TbpA | 10 μg |
| B only (IN) | TbpB | 10 μg |
| Control | PBS only | 0 |
| Sc A + B + Ctb | Ctb + TbpA + TbpB | 10 μg + 10 μg + 10 μg |
| (Sc^{b}) | admixed | |

^{a}Groups of mice (n = 5) were immunized three times at 10 day intervals.
^{b}One group was immunized subcutaneously with an admixture of TbpA, TbpB and Ctb.

ELISAs. Serum and vaginal washes were assayed for total and specific antibodies as described previously (34). For antibodies specific to Ctb, plates were first coated with 0.1 mL of GM1 ganglioside as described above. All capture antibodies and alkaline-phosphatase-conjugated goat-anti mouse isotype specific antibodies were purchased from Southern Biotechnology Associates (Birmingham, Ala.). The standard curve was generated using a mouse reference serum (Bethyl Laboratories).

Serum Bactericidal Assays. Mouse sera were pooled by group and heat inactivated at 56° C. for 30 min. Gonococcal strains were plated from freezer stocks directly onto GCB agar plates containing supplement 1 and 5 μM desferal to induce iron stress. For strains FA19 and FA1090, plates were allowed to incubate at 37° C.+5% CO₂ for approximately 24 hours, at which time they were passed again as above. Following the second passage, the plates were allowed to incubate for 16-18 hrs. Isolated colonies were picked from the plate and suspended in prewarmed 37° C. Gey's balanced salt solution (Sigma) containing 0.1% gelatin and 5 μM desferal (GBSS+G+D). The optical density at 600 nm of the inoculum was monitored until it reached 0.20 (0.23 for strain MS11), then it was serially diluted to 10⁻⁵ in prewarmed GBSS+G+D. Immediately following dilution, 80 μl of the diluted cell suspension was added to a prewarmed 96-well microtiter plate containing 10 μl of the appropriate serum samples diluted in GBSS+G+D. The plate was incubated at 37° C.+5% CO₂ for 15 min, then 10 μl of normal human pooled serum (Quidel Corp.) was added and the plate was again incubated as above for 45 min. After incubation, viable gonococci were detected by plating on GCB agar containing Kellogg's supplement 1 and 12.5 μM ferric nitrate. Plates were incubated for approximately 24 hours as described above, after which colonies were enumerated. Bactericidal titer was determined as the lowest dilution that gave >50% killing as compared to control sera at the same dilution. Strain MS11 was plated only once directly from the freezer stock and allowed to grow for 16-18 hrs. Because of its moderate sensitivity to human serum, bactericidal activity against strain MS11 was tested in 5% human sera, and incubated for 15 min.

Statistics. Analysis of variance for multiple group comparisons was performed using the Tukey-Kramer multiple-comparison test, or the Kruskal-Wallis multiple-comparison Z-value test where appropriate. A P value of <0.05 was considered significant.

### Results

Serum antibody responses against TbpA and TbpB. The serum antibody responses were measured over time using a quantitative ELISA, with which we measured antibody levels following each immunization. Sera were collected at day 0, 17, 28, 35, and 65. All day 0 sera were assayed and found to be negative for antibodies specific to all antigens tested. The antibody responses to TbpA and TbpB following vaccination were strikingly different and were dependant on antigen preparation and route of immunization. For TbpA, the highest antibody responses were seen in the Sc immunized group, in which antibodies to ThpA peaked on day 35 and remained high through day 65. The groups receiving ThpA conjugated to Ctb, and TbpA alone generated the next highest responses through day 28. Interestingly, the presence of Ctb in admixtures with ThpA appeared to delay the immune response against ThpA. However, by day 65, ThpA levels were similar for all IN immunized groups.

Unlike ThpA, conjugation of TbpB to Ctb significantly enhanced antibody titers, as compared to the groups where Ctb was admixed with TbpB (all comparisons P<0.05, days 17-65). Another important difference between ThpA and TbpB was that TbpB was poorly immunogenic when administered by itself, whereas TbpA alone was as immunogenic as the conjugated form). IN immunization with TbpB conjugate or with TbpB and TbpA conjugates together did not result in antibody levels that were significantly different from those elicited in the Sc-immunized group. In terms of antibody response, the groups immunized with both Tbps did not differ significantly from the group immunized with only one antigen at any day tested. Thus, although each antigen individually elicited distinct antibody responses, the presence of a co-administered antigen did not adversely effect antibody levels generated by IN vaccination. Antibody responses to Ctb were robust in all groups tested. Not surprisingly, the Sc immunized group elicited the highest Ctb antibody titers, except on day 65.

We were also able to detect serum IgA antibody responses specific for ThpA, TbpB and Ctb. Serum IgA levels against ThpA were transient, and not measureable until day 28, and completely undetectable in all groups by day 65. The low IgA levels detected against TbpA was probably reflective of this antigen's lower overall immunogenicity, as shown by lower serum IgG titers against ThpA as compared to TbpB. Serum IgA responses to TbpB were much higher than those measured against ThpA, with the highest detected serum IgA antibody responses found in the groups immunized with the TbpB-Ctb conjugates. The levels measured in the conjugate groups were not significantly different from one another, but were different from the only other groups with measurable serum IgA to TbpB: namely, the group immunized with ThpA+TbpB+Ctb and the subcutaneously immunized group (P<0.05, days 28-65). The Sc immunized animals had the highest serum IgG antibody titers against TbpB; however, the serum IgA titers elicited by this route were only detectable on day 65. Serum IgA titers to Ctb initially were the highest in the animals immunized with the TbpA-Ctb conjugate, followed by the other two conjugate groups. By day 65, all IN immunized groups had similar levels of Ctb-specific IgA antibody. Interestingly, on days 17 and 28 we were able to measure serum IgA to Ctb in the subcutaneously immunized group, but by day 35 Ctb-specific serum IgA was undetectable and remained so on day 65.

Vaginal antibody responses to ThpA and TbpB. The relative immunogenicities of ThpA and TbpB were also reflected in the detectable antibody levels measured in the vaginal washes. Vaginal wash antibodies detected on day 28 (seven days after the final immunization) to ThpA (Table 3) were not as robust as those detected against TbpB (Table 4). For ThpA-specific IgA (Table 3), on day 28 the highest response measured was generated by the group of animals immunized with the ThpA-Ctb conjugate: ThpA-specific IgA represented 1% of the total IgA antibody detected. This level however was only significantly different from the group immunized with both Thp conjugates (P<0.05) among the groups in which we were able to measure ThpA-specific IgA. Furthermore, only in the IN immunized groups were we able to detect ThpA-specific IgA. Interestingly, TbpA-specific IgA responses declined on day 35 in all groups with measurable IgA; however, these levels had returned to similar or slightly higher levels by day 65 (Table 3). Although the group receiving both TbpA and TbpB conjugates had increased antibody levels by day 65 they were still significantly lower than the groups immunized with ThpA-Ctb and with both Tbps admixed with Ctb (P<0.05) (Table 3). ThpA specific IgG levels were undetectable on day 28. We were unable to measure vaginal IgG until days 35 and 65 (Table 3). For the most part, vaginal IgG antibody levels specific for ThpA were lower and more sporadic in comparison to vaginal IgG measured to TbpB (Table 4).

TABLE 3
Vaginal antibody levels specific for TbpA, detected at days 28, 35, and 65^{a}.

| Immunization | Day 28^{b} | | Day 35 | | Day 65 | |
|---|---|---|---|---|---|---|
| groups | IgA | IgG | IgA | IgG | IgA | IgG |
| TbpA - Ctb | 1.0x/÷1.8 | 0 | 0.2x/÷1.7 | 0 | 0.9x/÷1.4 | 0.6x/÷1.7^{f} |
| TbpA - Ctb + | 0.1<^{c} | 0 | 0.1<^{d} | 0 | 0.3x/÷2.3 | 0 |
| TbpB - Ctb | | | | | | |
| TbpA + Ctb | 0 | 0 | 0 | 0 | 0 | 0.5x/÷1.3^{e} |
| TbpA + | 0.4x/÷6.3^{e} | 0 | 0.2x/÷3.8 | 0.6x/÷10.6^{d} | 1.5x/÷3.1 | 0.3x/÷2.7^{f} |
| TbpB + Ctb | | | | | | |
| TbpA | 0.5x/÷4.1^{e} | 0 | 0.2x/÷2.0 | 2.8x/÷3.8 | 0.6x/÷10.8^{e} | 0.2x/÷4.4^{g} |
| Sc TbpA + | 0 | 0 | 0 | 0 | 0 | 1.2′/, 1.3 |
| TbpB + Ctb | | | | | | |

^{a}Data are expressed as the geometric mean of the percentage of total corresponding antibody isotype concentrations x/÷ standard deviation
^{b}Day 28 is 7 days after final immunization.
^{c}Only one mouse had detectable TbpA-specific antibodies.
^{d}Only two mice had detectable TbpA-specific antibodies.
^{e}Only three mice had detectable TbpA-specific antibodies.
^{f}n = 4; one mouse removed due to very low total IgG.
^{g}n = 3; two mice removed due to very low total IgG

TABLE 4
Vaginal antibody levels specific for TbpB, detected at days 28, 35, and 65^{a}.

| Immunization | Day 28^{b} | | Day 35 | | Day 65 | |
|---|---|---|---|---|---|---|
| groups | IgA | IgG | IgA | IgG | IgA | IgG |
| TbpB - Ctb | 12.2x/÷2.9 | 32.5x/÷1.4^{f} | 6.4x/÷2.7 | 15.9x/÷1.5^{f} | 3.5x/÷1.7 | 2.0x/÷3.8^{d} |
| TbpA - Ctb + | 8.2x/÷3.6 | 20.2x/÷5.5 | 2.6x/÷2.9 | 20.4x/÷1.5 | 1.9x/÷2.9 | 9.7x/÷4.0^{e} |
| TbpB - Ctb | | | | | | |
| TbpB + Ctb | 0 | 0 | 0 | 0 | 0 | 0 |
| TbpA + | 0.2<^{c} | 0 | 0.1<^{d} | 1.8x/÷2.9^{e} | 0.1<^{d} | 1.6x/÷2.7^{c} |
| TbpB + Ctb | | | | | | |
| TbpB | 0.2<^{c} | 0 | 0 | 0.5x/÷1.3^{c,f} | 0 | 0 |
| Sc TbpA + | 0.1<^{d} | 20.5x/÷1.8 | 0.1<^{d} | 12.9x/÷1.5 | 0.1<^{d} | 15.8x/÷1.5 |
| TbpB + Ctb | | | | | | |

^{a}Data are expressed as the geometric mean of the percentage of total corresponding antibody isotype concentrations x/÷ standard deviation.
^{b}Day 28 is 7 days after final immunization.
^{c}Only one mouse had detectable TbpB-specific antibodies.
^{d}Only two mice had detectable TbpB-specific antibodies.
^{e}Only three mice had detectable TbpA-specific antibodies.
^{f}n = 4; one mouse removed due to very low total IgG.

In contrast to antibody levels measured to TbpA, TbpB-specific IgA and IgG levels were robust as early as day 28 (Table 4). Vaginal IgA levels specific for TbpB were highest in groups immunized with the Ctb conjugates, and were statistically different from the other IN immunized and subcutaneous groups (P<0.05 day 28) and remained significantly different through day 65 (P<0.05). The day 28 TbpB-specific IgG responses were also robust, with the highest levels measured in the IN immunization groups immunized with the Ctb conjugates and in the Sc group. In the IN groups immunized with Ctb admixed, we were unable to detect TbpB-specific IgG on day 28, however levels increased on subsequent days (Table 4), consistent with serum IgG increases. Though TbpB-specific levels of IgA and IgG were initially robust in the groups immunized with the TbpB-Ctb conjugate, they were in decline by day 65 (Table 4).

Vaginal antibody responses to Ctb were also robust, and generally higher than those responses measured against ThpA or TbpB (data not shown). This is presumably reflective of this antigen's higher immunogenicity and is consistent with higher serum IgG levels. The groups immunized with the Ctb conjugates, as opposed to the admixtures, generally induced the highest Ctb-specific antibody responses. Interestingly, the Sc group had high levels of Ctb specific IgA, whereas Sc immunization with the Tbps, resulted in IgA levels that were almost zero (data not shown).

Serum bactericidal activity. In order to determine whether serum antibodies had bactericidal activity, we performed in vitro serum bactericidal assays using pooled mouse serum from day 35, and human serum as a complement source. The data demonstrate that those animals immunized with both Thp-Ctb conjugates had the greatest bactericidal activity against both homologous and heterologous strains tested (Table 5). The sera from the group immunized with the TbpA-Ctb conjugate was more effective at killing the homologous strain (FA19) and one heterologous strain (MS11) than was the TbpB-Ctb sera. This outcome is interesting considering the significantly lower serum antibody titers generated against ThpA, in comparison to those generated against TbpB. The Sc group had the highest ThpA and TbpB antibody titers on day 35; however, these sera were the least bactericidal against the homologous strain of all the groups tested. Furthermore, sera from this group were the only ones that failed to show bactericidal activity against any of the heterologous strains tested.

TABLE 5
Serum bactericidal activity of sera collected at day 35.
Serum bactericidal titre^{a}

| | Strains | | | | |
|---|---|---|---|---|---|
| Immunization group | FA19 | FA1090 | | MS11 | |
| TbpA - Ctb + TbpB - Ctb | 800 (84% ± 5.7) | 200 | (64% ± 1.4) | 400 | (60% ± 0.7) |
| TbpA - Ctb | 400 (83% ± 4.2) | 25 | (<50%)^{b} | 200 | (64% ± 1.4) |
| TbpB - Ctb | 200 (64% ± 1.4) | 25 | (<50%)^{b} | 50 | (56% ± 4.9) |
| Sc TbpA + TbpB + Ctb | 100 (70% ± 1.4) | 25 | (<50%)^{b} | 25 | (78%)^{b} |

| | | | |
|---|---|---|---|
| TbpA only | 400 (71% ± 9.9) | ND^{c} | ND^{c} |

^{a}Data are represented as the lowest reciprocal dilution that gave >50% killing. The average percent killing determined from duplicate assays ± standard deviation is shown in parentheses.
^{b}Assays conducted at 1/25 dilutions were performed only once and lower dilutions were not tested.
^{c}ND, not determined.

IgG subclass analysis. We performed IgG subclass analysis on selected serum samples in an effort to gain insight into why some serum pools performed better than others in bactericidal assays. It has been shown previously that mouse IgG2a is the most efficient IgG subclass in activating complement, while IgG1 is poor and possibly may be inhibitory (16, 27,44). We found that animals immunized with the ThpA-Ctb conjugate had higher IgG2a antibody responses, and hence a lower IgG1/IgG2a ratio. Those animals immunized with the TbpB-Ctb conjugate produced significantly more IgG1 than IgG2a antibodies and had a very high IgG1/IgG2a ratio. Interestingly, in the animals immunized simultaneously with both Ctb conjugates, the presence of ThpA and TbpB in the same antigen preparation influenced the IgG1 to IgG2a ratio of antibodies elicited against the individual antigens. The presence of ThpA increased the level of IgG2a to TbpB, whereas the presence of TbpB resulted in increased production of IgG1 and decreased levels of IgG2a against ThpA. Contrary to expectations, the Sc immunized animals had low IgG1/IgG2a ratios against both ThpA and TbpB; however, as demonstrated above, sera from this group performed the poorest in terms of bactericidal activity.

### Discussion

Previous studies have shown IN immunization to be an effective means for the induction of serum and mucosal adjuvant-specific antibodies (21, 23, 24, 48, 49). The induction of prolonged genital tract antigen-specific antibodies following IN vaccination has highlighted this route of immunization as an attractive potential method for preventing STIs (37, 47). We explored this possibility by immunizing mice IN with recombinant transferrin binding proteins A and/or B in conjunction with the mucosal adjuvant cholera toxin B. We demonstrate that IN immunization with these antigens is an effective means of eliciting specific, serum and vaginal anti-Thp antibodies. However, each Thp antigen behaved differently in regards to overall immunogenicity.

TbpB was the more immunogenic of the two proteins. Large differences in immunogenicity between the antigens were apparent regardless of the route of immunization. IN immunization elicited the highest anti-TbpB titers when TbpB was conjugated to Ctb. Admixing TbpB with Ctb improved the immunogenicity of TbpB over control groups; however, differences between admixed groups and those in which TbpB was conjugated to Ctb were statistically significant, in general. TbpB was poorly immunogenic if administered alone, in the absence of the Ctb adjuvant. By contrast, maximal ThpA-specific serum antibody responses following IN immunization were not dependent on the presence of Ctb. Mice immunized IN with ThpA alone elicited serum antibody titers similar to those generated by the group immunized with the TbpA-Ctb conjugate. This may have been the result of the inclusion of the non-ionic detergent, lauryl maltoside, to the ThpA antigen preparations. Lauryl maltoside has been shown to act as an absorption enhancer in the nasal cavity (1, 32). This may have allowed better absorption of TbpA, as large molecular weight proteins are usually poorly absorbed in the nasal cavity without enhancers (36). On the other hand, the relatively poor immunogenicity of TbpB administered alone was likely due to the solubility of the TbpB used in this study. Native TbpB is a lipoprotein, and anchored to the bacterial outer membrane via a lipid tail, and contains no predicted transmembrane segments. To simplify recombinant protein expression and purification, we expressed TbpB in E. coli without the amino-terminal cysteine, where lipidation normally occurs. Because of its overall hydrophilicity, it is likely that over-expressed, lipid-free TbpB would have been excluded from detergent micelles (22). The enhanced immunomodulatory effects with TbpB conjugated to Ctb therefore are likely due in part to binding of Ctb to GM1 ganglioside on nasal mucosa cells, which is thought to enhance antigen uptake and presentation to the immune system.

Interestingly, Ctb admixed with ThpA delayed generation of antibodies against TbpA as shown by the statistically significant differences in antibody titers measured on days 17 and 28. However this effect was abrogated by day 65, at which time there were no significant differences in the levels of ThpA-specific antibody titers against ThpA among any of the IN immunized groups. The concurrent IN immunization with both TbpA and TbpB did not have a negative effect on levels of antibodies to either antigen when compared to groups where each antigen was administered alone; however the IgG subclass distribution was influenced by the presence of either antigen. These alterations in IgG subclass distribution however did not appear to be deleterious, as the bactericidal activity of pooled sera from the group immunized with both ThpA and TbpB was superior to those sera from animals immunized with a single antigen. This demonstrates that both antigens can be administered simultaneously without negatively influencing antibody levels or serum bactericidal activity.

Similar to the situation with specific antibody levels in the serum, vaginal antibody responses to TbpB were generally much higher than those elicited against ThpA. The robust genital tract TbpB-specific antibody responses measured were also dependent on conjugation to Ctb, whereas this was not the case with ThpA. Immunization with ThpA elicited mostly IgA, while measurable IgG responses were low and sporadic. The low levels of ThpA-specific vaginal IgG were not surprising, as it is thought that most vaginal IgG originates from serum transudation (41). Although ThpA IgA levels were low, they remained mostly steady through day 65, except for a transient decrease on day 35. It is possible this decrease could have resulted from the mouse estrus cycle, as levels of genital tract immunoglobulins fluctuate during the cycle (37). The levels of TbpB-specific IgA and IgG, though initially robust, decreased significantly during the course of the study. This decline in antibody levels over time is not uncommon. Wu et al. followed the genital tract antibody levels in IN immunized mice for a one year period (47). They demonstrated that by 4 months post immunization, antibody levels had decreased extensively from their initial analysis, but appeared to level out throughout the course of one year (47). The aim of the current study was not to characterize the duration of anti-Thp immune responses, but future studies will address the longevity of the antibody response following IN immunization, and whether these immune responses are protective.

We performed serum bactericidal assays as a correlate for the induction of protective antibody responses. We detected serum bactericidal activity against the homologous gonococcal strain (FA19) and two heterologous strains (FA1090 and MS11) using human serum as a complement source. We found that all IN immunization groups yielded sera with greater bactericidal activity than the Sc immunized group. The group immunized IN with both ThpA and TbpB gave the highest serum bactericidal titers, and was the only pool of serum that contained bactericidal antibodies reactive against all three strains tested. Surprisingly, the group immunized IN with ThpA elicited the second-highest bactericidal titers, in spite of the fact that serum IgG levels were approximately 20-fold lower than TbpB titers at that time point. This suggests that ThpA may be the more ideal target in the development of a vaccine. Studies have shown that ThpA is the more conserved of the two proteins (11, 12), which may be why this antigen elicited sera with more cross-bactericidal activity. Furthermore, in a meningococcal vaccine study, mice immunized with TbpA or ThpA and TbpB were completely protected following lethal challenge, but the group immunized with TbpB only was not (46).

The obvious discrepancies between antibody titers and serum bactericidal activities suggested that qualitative rather than quantitative differences existed among antibody preparations, which prompted us to perform IgG subclass analysis. We found that those animals immunized IN with the ThpA-Ctb conjugate elicited higher levels of IgG2a than did those animals immunized with the TbpB-Ctb conjugate. In mice, the IgG2a isotype is the most efficient activator of complement, while the IgG1 isotype is the poorest complement activator (16, 27, 44). Thus the lower IgG1:IgG2a ratio detected could in part explain the enhanced bactericidal activity observed with the ThpA anti-serum. The results of the IgG subclass analysis do not, however, explain why the Sc group, immunized with both Tbps, differed so dramatically from its IN immunized counterpart in terms of bactericidal activity. The IgG1:IgG2a ratios against TbpA for both IN and Sc groups were similar (0.5 and 0.8). By contrast, the IgG1:IgG2a ratio against TbpB in the Sc group was nearly three times lower than that of the IN group (6.0 and 17.9). In spite of this, the bactericidal activity of the Sc immunized group was comparatively poor. These results suggest that antigens delivered by IN immunization may better retain a native conformation compared to subcutaneous immunization. Bactericidal activity is associated with high-avidity antibodies, elicitation of which correlates with the ability to keep protein antigens in native conformation (9). Furthermore, vaccine studies using meningococcal PorA demonstrated that PorA is immunogenic when administered via Sc immunization in conjunction with a variety of adjuvants; however, only mice immunized with PorA contained in outer membrane vesicles or liposomes generated antibodies with bactericidal activity (2, 9). This suggests that antigens delivered Sc may be subject to misfolding or possibly proteolysis unless protected in a membrane. By contrast, the current study indicates that protein degradation or misfolding, resulting in non-native presentation, may not be as problematic if the antigens are delivered intranasally.

Whether bactericidal activity is an important mediator of immunity in the genital tract is a matter of speculation. Though complement lytic activity has been demonstrated previously in human cervical mucus (35), complement levels are highly variable among individuals and are influenced by hormonal cycles (43). Furthermore, IgA levels are high in the female genital tract, and IgA has been shown to be inhibitory to IgG complement activation (39). Therefore, bactericidal activity in the female genital tract may not be an important mediator of protection. Mucosal IgA has been shown to be important in the protection of the mucosal surfaces from invading bacteria, viruses, and toxins (39). Furthermore, studies have shown enhanced protective abilities of polymeric IgA as compared to IgG in passive protective studies in mice (39). The precise role of the different antibody isotypes in protection of the genital tract remains to be elucidated. However, studies performed in mice have shown protection against Chlamydia trachomatis genital infection is attributable to IgA. Cui et al. showed that following immunization and subsequent Chlamydia challenge, clearance of cervical chlamydial antigen correlated with increases in cervical IgA, but not in IgG (15). Furthermore, Pal et al. showed that a monoclonal IgA antibody against the chlamydial major outer membrane protein could confer passive protection in mice (31). Finally, in humans, levels of IgA in vaginal secretions and the amount of C. trachomatis isolated from the cervix are inversely correlated (7). In a recent study, mice immunized IN with gonococcal outer membrane preparations resulted in strong serum bactericidal activity and decreased gonococcal vaginal colonization of estradiol-treated mice (33). This study also showed that antigen-specific IgA titers were 8-16 fold higher than IgG titers in the mice with reduced vaginal colonization (33). These studies suggest that IgA may be more important than IgG for protection against bacterial sexually transmitted infections, and highlights the importance of inducing genital IgA following vaccination.

In conclusion, we have demonstrated induction of both serum and vaginal bactericidal antibodies in a mammal following IN immunization with chemically conjugated proteins comprising ThpA, TbpB or both. This example thus illustrates the utility of linking or co-administering ThpA and TbpB in order to elicit a strong and effective immune response to these antigens.

### REFERENCES FOR EXAMPLE 1

1. Ahsan, F., J. J. Arnold, E. Meezan, and D. J. Pillion. 2001. Mutual inhibition of the insulin absorption-enhancing properties of dodecylmaltoside and dimethyl-beta-cyclodextrin following nasal administration. Pharm Res. 18:608-14.
2. Arigita, C., G. F. Kersten, T. Hazendonk, W. E. Hennink, D. J. Crommelin, and W. Jiskoot. 2003. Restored functional immunogenicity of purified meningococcal PorA by incorporation into liposomes. Vaccine. 21:950-60.
3. Bergquist, C., E. L. Johansson, T. Lagergard, J. Holmgren, and A. Rudin. 1997. Intranasal vaccination of humans with recombinant cholera toxin B subunit induces systemic and local antibody responses in the upper respiratory tract and the vagina. Infect. Immun. 65:2676-2684.
4. Boslego, J. W., E. C. Tramont, R. C. Chung, D. G. McChesney, J. Ciak, J. C. Sadoff, M. V. Piziak, J. D. Brown, C. C. Brinton, Jr., S. W. Wood, and et al. 1991. Efficacy trial of a parenteral gonococcal pilus vaccine in men. Vaccine. 9:154-162.
5. Bouvet, J. P., L. Belec, R. Pires, and J. Pillot. 1994. Immunoglobulin G antibodies in human vaginal secretions after parenteral vaccination. Infect Immun. 62:3957-61.
6. Broliden, K., J. Hinkula, C. Devito, P. Kiama, J. Kimani, D. Trabbatoni, J. J. Bwayo, M. Clerici, F. Plummer, and R. Kaul. 2001. Functional HIV-1 specific IgA antibodies in HIV-1 exposed, persistently IgG seronegative female sex workers. Immunol Lett. 79:29-36.
7. Brunham, R. C., C. C. Kuo, L. Cles, and K. K. Holmes. 1983. Correlation of host immune response with quantitative recovery of Chiamydia trachomatis from the human endocervix. Infect Immun. 39: 1491-4.
8. Centers for Disease Control and Prevention. 2001. Sexually Transmitted Disease Surveillance, 2001. U.S. Department of Health and Human Services. Centers for Disease Control and Prevention, Atlanta, Ga.
9. Christodoulides, M., J. L. Brooks, E. Rattue, and J. E. Heckels. 1998. Immunization with recombinant class I outer-membrane protein from Neisseria meningitidis: influence of liposomes and adjuvants on antibody avidity, recognition of native protein and the induction of a bactericidal immune response against meningococci. Microbiology. 144:3027-37.
10. Cohen, M. S., I. F. Hoffman, R. A. Royce, P. Kazembe, J. R. Dyer, C. C. Daly, D. Zimba, P. L. Vemazza, M. Maida, S. A. Fiscus, and J. J. Eron, Jr. 1997. Reduction of concentration of HIV-1 in semen after treatment of urethritis: implications for prevention of sexual transmission of HIV-1. AIDSCAP Malawi Research Group. Lancet. 349:1868-1873.
11. Cornelissen, C. N., J. E. Anderson, I. C. Boulton, and P. F. Sparling. 2000. Antigenic and sequence diversity in gonococcal transferrin-binding protein A. Infect. Immun. 68:4725-4735.
12. Cornelissen, C. N., J. E. Anderson, and P. F. Sparling. 1997. Characterization of the diversity and the transferrin-binding domain of gonococcal transferrin-binding protein 2. Infect. Immun. 65:822-828.
13. Cornelissen, C. N., G. D. Biswas, and P. F. Sparling. 1993. Expression of gonococcal transferrin-binding protein 1 causes Escherichia coli to bind human transferrin. J. Bacteriol. 175:2448-2450.
14. Cornelissen, C. N., M. Kelley, M. M. Hobbs, J. E. Anderson, J. G. Cannon, M. S. Cohen, and P. F. Sparling. 1998. The transferrin receptor expressed by gonococcal strain FA1090 is required for the experimental infection of human male volunteers. Mol. Microbiol. 27:611-616.
15. Cui, Z. D., D. Tristram, L. J. LaScolea, T. Kwiatkowski, Jr., S. Kopti, and P. L. Ogra. 1991. Induction of antibody response to Chlamydia trachomatis in the genital tract by oral immunization. Infect Immun. 59:1465-9.
16. Ey, P. L., G. J. Russell-Jones, and C. R. Jenkin. 1980. Isotypes of mouse IgG-I. Evidence for ‘non-complement-fixing’ IgG1 antibodies and characterization of their capacity to interfere with IgG2 sensitization of target red blood cells for lysis by complement. Mol. Immunol. 17:699-710.
17. Farley, T. A., D. A. Cohen, and W. Elkins. 2003. Asymptomatic sexually transmitted diseases: the case for screening. Prev Med. 36:502-9.
18. Gallichan, W. S., and K. L. Rosenthal. 1995. Specific secretory immune responses in the female genital tract following intranasal immunization with a recombinant adenovirus expressing glycoprotein B of herpes simplex virus. Vaccine. 13:1589-95.
19. Gerbase, A. C., J. T. Rowley, and T. E. Mertens. 1998. Global epidemiology of sexually transmitted diseases. Lancet. 351:2-4.
20. Greenberg, L., B. B. Diena, F. A. Ashton, R. Wallace, C. P. Kenny, R. Znamirowski, H. Ferrari, and J. Atkinson. 1974. Gonococcal vaccine studies in Inuvik. Can. J. Public Health. 65:29-33.
21. Hajishengallis, G., S. K. Hollingshead, T. Koga, and M. W. Russell. 1995. Mucosal immunization with a bacterial protein antigen genetically coupled to cholera toxin A2/B subunits. J. Immunol. 154:4322-4332.
22. Helenius, A., and K. Simons. 1975. Solubilization of membranes by detergents. Biochim Biophys Acta. 415:29-79.
23. Isaka, M., Y. Yasuda, S. Kozuka, Y. Miura, T. Taniguchi, K. Matano, N. Goto, and K. Tochikubo. 1998. Systemic and mucosal immune responses of mice to aluminium-adsorbed or aluminium-non-adsorbed tetanus toxoid administered intranasally with recombinant cholera toxin B subunit. Vaccine. 16:1620-6.
24. Isaka, M., Y. Yasuda, M. Mizokami, S. Kozuka, T. Taniguchi, K. Matano, J. Maeyama, K. Mizuno, K. Morokuma, K. Ohkuma, N. Goto, and K. Tochikubo. 2001. Mucosal immunization against hepatitis B virus by intranasal co-administration of recombinant hepatitis B surface antigen and recombinant cholera toxin B subunit as an adjuvant. Vaccine. 19:1460-6.
25. Ison, C. A., J. A. Dillon, and J. W. Tapsall. 1998. The epidemiology of global antibiotic resistance among Neisseria gonorrhoeae and Haemophilus ducreyi. Lancet. 351:8-11.
26. Kenney, C. D., and C. N. Cornelissen. 2002. Demonstration and characterization of a specific interaction between gonococcal transferrin binding protein A and TonB. J. Bacteriol. 184:6138-6145.
27. Koolwijk, P., J. H. Boot, R. Griep, and B. J. Bast. 1991. Binding of the human complement subcomponent C1q to hybrid mouse monoclonal antibodies. Mol Immunol. 28:567-76.
28. Meyer, T. F., J. Pohlner, and J. P. van Putten. 1994. Biology of the pathogenic Neisseriae. Curr. Top. Microbiol. Immunol. 192:283-317.
29. Mickelsen, P. A., and P. F. Sparling. 1981. Ability of Neisseria gonorrhoeae, Neisseria meningitidis, and commensal Neisseria species to obtain iron from transferrin and iron compounds. Infect. Immun. 33:555-564.
30. Nardelli-Haefliger, D., R. Roden, C. Balmelli, A. Potts, J. Schiller, and P. De Grandi. 1999. Mucosal but not parenteral immunization with purified human papillomavirus type 16 virus-like particles induces neutralizing titers of antibodies throughout the estrous cycle of mice. J Virol. 73:9609-13.
31. Pal, S., I. Theodor, E. M. Peterson, and L. M. de la Maza. 1997. Monoclonal immunoglobulin A antibody to the major outer membrane protein of the Chlamydia trachomatis mouse pneumonitis biovar protects mice against a chlamydial genital challenge. Vaccine. 15:575-82.
32. Pillion, D. J., S. Hosmer, and E. Meezan. 1998. Dodecylmaltoside-mediated nasal and ocular absorption of lyspro-insulin: independence of surfactant action from multimer dissociation. Pharm Res. 15:1637-9.
33. Plante, M., A. Jerse, J. Hamel, F. Couture, C. R. Rioux, B. R. Brodeur, and D. Martin. 2000. Intranasal immunization with gonococcal outer membrane preparations reduces the duration of vaginal colonization of mice by Neisseria gonorrhoeae. J. Infect. Dis. 182:848-855.
34. Price, G. A., M. M. Hobbs, and C. N. Cornelissen. 2004. Immunogenicity of gonococcal transferrin binding proteins during natural infections. Infect. Immun. 72:277-283.
35. Price, R. J., and B. Boettcher. 1979. The presence of complement in human cervical mucus and its possible relevance to infertility in women with complement-dependent sperm-immobilizing antibodies. Fertil Steril. 32:61-6.
36. Quraishi, M. S., N. S. Jones, and J. D. Mason. 1997. The nasal delivery of drugs. Clin Otolaryngol. 22:289-301.
37. Rosenthal, K. L., and W. S. Gallichan. 1997. Challenges for vaccination against sexually-transmitted diseases: induction and long-term maintenance of mucosal immune responses in the female genital tract. Semin Immunol. 9:303-14.
38. Rudin, A., G. C. Riise, and J. Holmgren. 1999. Antibody responses in the lower respiratory tract and male urogenital tract in humans after nasal and oral vaccination with cholera toxin B subunit. Infect Immun. 67:2884-90.
39. Russell, M. W., and M. Kilian. 2005. Biological Activities of IgA, p. 267-289. In Mestecky, J., J. Bienenstock, M. E. Lam, M L. Mayer, W. Strober, and J. R. McGhee (ed.). Mucosal Immunology, Third Edition. Elsevier/Academic Press, San Diego.
40. Russell, M. W., P. F. Sparling, R. P. Morrison, S. Cauci, P. L. Fidel, D. Martin, E. W. Hook, J. Mestecky. 2005. Mucosal immunology of sexually transmitted diseases, p. 1693-1720. In Mestecky, J., J. Bienenstock, M. E. Lam, L. Mayer, W. Strober, J. R. McGhee (ed.), Mucosal immunology, Third Edition. Elsevier/Academic Press, San Diego.
41. Russell, M. W., and J. Mestecky. 2002. Humoral immune responses to microbial infections in the genital tract. Microbes Infect. 4:667-77.
42. Russell, M. W., Z. Moldoveanu, P. L. White, G. J. Sibert, J. Mestecky, and S. M. Michalek. 1996. Salivary, nasal, genital, and systemic antibody responses in monkeys immunized intranasally with a bacterial protein antigen and the cholera toxin B subunit. Infect. Immun. 64:1272-1283.
43. Schumacher, G. F. 1988. Immunology of spermatozoa and cervical mucus. Hum Reprod. 3:289-300.
44. Seino, J., P. Eveleigh, S. Warnaar, L. J. van Haarlem, L. A. van Es, and M. R. Daha. 1993. Activation of human complement by mouse and mouse/human chimeric monoclonal antibodies. Clin Exp Immunol. 94:291-6.
45. Tramont, E. C. 1989. Gonococcal vaccines. Clin. Microbiol. Rev. 2 Suppl:S74-S77.
46. West, D., K. Reddin, M. Matheson, R. Heath, S. Funnell, M. Hudson, A. Robinson, and A. Gorringe. 2001. Recombinant Neisseria meningitidis transferrin binding protein A protects against experimental meningococcal infection. Infect. Immun. 69:1561-1567.
47. Wu, H. Y., S. Abdu, D. Stinson, and M. W. Russell. 2000. Generation of female genital tract antibody responses by local or central (common) mucosal immunization. Infect Immun. 68:5539-45.
48. Wu, H. Y., and M. W. Russell. 1998. Induction of mucosal and systemic immune responses by intranasal immunization using recombinant cholera toxin B subunit as an adjuvant. Vaccine. 16:286-92.
49. Wu, H. Y., and M. W. Russell. 1993. Induction of mucosal immunity by intranasal application of a streptococcal surface protein antigen with the cholera toxin B subunit. Infect Immun. 61:314-22.

### Example 2

### Chimeric Proteins Comprising Single Domains of TbpA and TbpB and a Mucosal Adjuvant

One potential drawback with using the full-length Thp proteins is their intrinsic ability to bind to human transferrin. By binding to transferrin, protective epitopes necessary to elicit protective antibodies could become blocked or misfolded. The resultant antibody titers could therefore be below protective levels, or unable to recognize Tbps on the bacterial surface. To circumvent this problem, epitopes that cannot bind to transferrin but can elicit protective antibodies could be used in place of the full-length proteins.

In an effort to determine whether specific domains of the transferrin binding proteins could elicit protective antibodies, we made genetic chimeras using single domains from ThpA and TbpB, linked to the A2 subunit of cholera toxin and E. coli heat-labile toxin IIb. For ThpA we focused on the surface exposed loop 2 (L2), the amino acid sequence of which is presented in FIG. 4B (SEQ ID NO: 7). For TbpB, we focused on the so-called high affinity N-terminal binding domain (NB), the sequence of which is presented in FIG. 4A (SEQ ID NO: 6). Although this domain retains the ability to bind transferrin as demonstrated by Western blot, this study wanted to determine whether this domain is immunogenic, and can elicit bactericidal antibodies.

Because L2 is relatively small in size (9 KDa), and from a protein that is poorly immunogenic we constructed a double genetic chimera. The strategy we employed expressed NB and L2 together in hopes that the larger NB would be more immunogenic and help to augment antibody responses to L2. To this end, we genetically linked the L2 in frame and immediately downstream of NB to make an NB-L2-A2 chimera. This approach also afforded us the opportunity to determine what effects the inclusion of both epitopes would have on bactericidal killing compared to mice immunized with only NB. In this study, we immunized mice intranasally and parenterally with our chimeric molecules. In addition to using Ctb chimeras, we also included LtbIIb chimeras for analysis, in an effort to compare the immune potentiating ability of the two. The results showed that both Ctb and LtbIIb chimeras are immunogenic, eliciting serum antibodies to NB and L2, and vaginal antibodies to NB. However, the chimeric antibody responses were not as robust as the full-length proteins using Ctb as adjuvants, as in Example 1. Though chimeric antibody responses were lower than those to the full-length proteins, the chimeras induced superior bactericidal responses against both homologous and heterologous strains. This study highlights the potential of using epitopes instead of full-length Tbps in eliciting protective immune responses.

### Cloning of Thp-Ctb and Thp-LtbIIb Chimeras

The original Ctb chimeric expression plasmid, pCT^{ΔA1} was previously described (Hajishengallis, G., S. K. Hollingshead, T. Koga, and M. W. Russell. 1995. Mucosal immunization with a bacterial protein antigen genetically coupled to cholera toxin A2/B subunits. J. Immunol. 154:4322-4332.). It consists of the tandem cholera toxin a2 and ctb genes under the control of a T7 promoter. Immediately, upstream of the a2 gene is a multiple cloning site consisting of NcoI and XhoI restriction sites. This multiple cloning site allows for the directional cloning of a gene of interest, genetically incorporated immediately upstream of the a2 gene. Therefore, once this construct is expressed in E. coli, the protein of interest is covalently fused to the A2 domain of the cholera toxin A subunit. The plasmid also contains a pelB leader sequence immediately upstream of the MCS that allows for periplasmic protein transport. The ctb gene contains the full-length cholera toxin B subunit gene, including the native V. cholerae signal sequence.

Once expression of the plamid is induced in E. coli, one mRNA transcript is made. This single transcript contains two ribosomal binding sites (RBS), one for the chimeric/A2 protein, and one for the cholera toxin B protein (Ctb). Both proteins once translated, are individually transported to the periplasm where assembly of the Ctb chimera occurs. Ctb forms a pentameric ring-like structure composed of 5 individual B subunits non-covalently attached. During B subunit assembly, one A2 subunit is inserted via its C-terminal end into the hole formed by the B subunit. This allows for a natural, non-covalent association of a protein of interest into Ctb. This expression and assembly process is the same for the E. coli heat labile enterotoxins, which share homology with cholera toxin.

Construction of the NB-Ctb chimera involved the amplification of the N-terminal binding domain of the tbpB gene from FA19 genomic DNA. This PCR product flanked by NcoI and XhoI sites was eventually cloned into pCT^{ΔA1}, creating the expression plasmid pVCU721. To aid in the purification of this chimeric molecule, a new expression plamid was constructed that incorporated a histidine tag into the C-terminus of the Ctb molecule. To this end, a Ctb expression plasmid was constructed by amplifying the mature ctb gene product from plasmid pCT^{ΔA1}. The PCR product was ultimately inserted into the expression plasmid pET-22b(+) in frame with a six histidine coding region so the resultant plasmid expressed Ctb monomers containing a histidine tag. To make the NB-Ctb(His) chimera expression plasmid, pVCU722, an internal NdeI restriction site within the ctb coding region was utilized. Plasmids pVCU720 and pVCU721 were restriction digested with NcoI and NdeI. This restriction digest liberated a pelB-nB-a2-ctbss (partial signal sequence) gene fragment from pVCU720. Similar digestion of pVCU721 linearized this plasmid immediately upstream of the pelB signal sequence which was in-frame with the mature ctb gene. Ligation of these fragments generated the NB-Ctb(his) chimeric expression plasmid pVCU722.

As mentioned above, the loop 2 domain (L2) from ThpA was also incorporated into the NB expression chimeras. For the Ctb chimera, L2 was amplified from FA19 genomic DNA using primers containing XhoI restriction sites. The forward primer also had an internal BamHI restriction site engineered into the primer to determine directionality of L2 insert in subsequent clones using restriction digestion. This scheme yielded the NB-L2-Ctb expression plasmid pVCU724. To obtain the NB-L2-Ctb(his) expression plasmid, pVCU724 and pVCU721 were digested with NcoI and NdeI. This liberated a pelB-nB-12-a2-ctbss (partial signal sequence) gene fragment from pVCU724 which was ligated into pVCU721 to yield the NB-L2-Ctb(his) expression vector pVCU725.

To generate the LtbIIb(his) chimeras, the expression plasmid pHN3.1 was utilized. This construct already contained a six histidine tag at the C-terminus of the LtbIIb gene, as well as the salivary binding region (SBR) from Streptococcus mutans upstream of the A2 subunit of LT-IIb. To create the NB-LtbIIb(his) expression construct, pHN3.1 was digested with NcoI/XhoI to liberate the sbr gene. The original NB PCR product used to make pVCU720 was ligated into pHN3.1 to generate the NB-LtbIIb(his) expression plasmid pVCU723. To create the NB-L2-LtbIIb(his) expression construct, pVCU723 was linearized with XhoI and the 12 gene product was used to create pVCU726.

### Expression and Purification of Ctb(his) and LtbIIb(his) Chimeras

The E. coli expression strain C41 (DE3) (Avidis), was transformed with each of the his-tagged chimeras described above. Following overnight induction in the presence (Ctb chimeras) or absence (Ltb chimeras) of IPTG, cell pellets were extracted to remove periplasmic cellular components. The cellular periplasmic components were subjected to ammonium sulfate precipitation, and ultimately resuspended in phoshphate buffer. The resuspended periplasmic fractions were batch-bound to nickel resin for affinity purification. Following nickel affinity purification, eluted fractions were analyzed by SDS-PAGE and Western blot.

Both NB and NB-L2 Ctb chimeras produced intact Ctb pentamers that were resistant to SDS dissociation by SDS-PAGE. The Ctb pentamer migrates at a molecular weight of approximately 52 kDa as demonstrated in the non-heated, non-reduced samples. After boiling, this pentameric structure is disrupted and the monomers migrate at molecular weight of approximately 12 kDa. Though, Ctb is resistant to the effects of SDS, the A subunit is not, and dissociates from the B subunit upon SDS-PAGE. The A1 subunit of Cholera toxin has a predicted molecular weight of approximately 27 kDa, and migrated accordingly. The NB-A2 and NB-L2-A2 chimeras have a predicted molecular weight of 53 and 61 kDa respectively. As view by SDS-PAGE, the chimeric NB molecules ran as doublets of apparent similar molecular weight, even after heating. The shift in molecular weight between the two chimeric molecules wasn't apparent by Coomasie staining but was obvious in Western blots using both TbpB and ThpA specific antiserum. Interestingly, the anti-ThpA sera was highly reactive with a band of ˜22 kDa, though no band was apparent by Coomasie blue staining. This band is likely L2-A2, which has a predicted molecular weight of ˜17 kDa. Since this band was not apparent by Coomasie staining, yet was the most robust by Western blot, suggests the proximity of the NB subunit to L2 may somehow interfere with anti-L2 antibody recognition.

As with Ctb, the LtbIIb chimeras also produced intact B subunits which were dissociated into monomers by heating. The B subunits of LtbIIb are similar in size to the B subunits of cholera toxin. The Ltb chimeras also produced both the NB-A2 and NB-L2-A2 chimeras as demonstrated by Western blot. Again, as with the NB-L2-Ctb chimera, a robust band was demonstrated on Western blot at ˜22 kDa in the LtbIIb background, which was not apparent on a Coomasie stained gel.

Both NB-A2 and NB-L2 chimeras were unstable, demonstrating various breakdown species as shown in both SDS-PAGE and Western blot analyses. This result was not unexpected, as TbpB and TbpB peptides have been previously been shown to be unstable, producing breakdown products following SDS-PAGE. In regards to toxin stoichiometry, chimeric-A2 and B subunit band intensities demonstrated a large amount of free B subunits. The exception to this was the NB-Ctb chimera which had similar band intensities as CT. Issues with stoichiometry are complicated however due to the propensity of NB to breakdown following SDS-PAGE, which could falsely demonstrate poor assembly. Assembly of functional chimeras however was established by GM1 and GD1a ganglioside ELISA (Data not shown).

### Vaccination Schedule

Groups of five female BALB/c mice at approximately 12 weeks of age were vaccinated for this study. Four groups were primed with one of the four chimeras intranasally, and two groups were primed subcutaneously with either NB-L2-Ctb or NB-L2-LtbIIb (Table 6). Each group was primed three times with a 10-day interval between vaccinations. Twenty-two days following the final priming vaccination, all groups were boosted two times with a 10-day interval between vaccinations. For the boosting vaccinations, all groups that received the chimeras were boosted intraperitoneally (IP).

TABLE 6
Immunization groups

| | | Amt |
|---|---|---|
| Group | | administered^{b} |
| (immunization route^{a}) | Immunogen | (μg) |
| NB-Ctb (IN/IP) | NB-Ctb chimera | 20 |
| NB-L2-Ctb (IN/IP) | NB-L2-Ctb chimera | 20 |
| NB-LtbIIb (IN/IP) | NB-LtbIIb chimera | 20 |
| NB-L2-LtbIIb (IN/IP) | NB-L2-LtbIIb chimera | 20 |
| NB-L2-Ctb (s.c./IP) | NB-L2-Ctb chimera | 20 |
| NB-L2-LtbIIb (s.c./IP) | NB-L2-LtbIIb chimera | 20 |
| Control (IN/IP) | Buffer 0 | 0 |

^{a}Mice were immunized three times either intranasally (IN) or subcutaneously (s.c.), followed by 2 boosts given intraperitonealy (IP).
^{b}Groups of mice (n = 5) were immunized three times at 10 day intervals. Twenty-two days following the third vaccination, the mice were boosted two times at 10-day intervals

### Serum Antibody Responses

Serum antibody levels were measured against full-length TbpA and TbpB at various time points using a quantitative ELISA. Following the three initial immunizations, antibody responses to TbpB in all groups immunized with the chimeras were low compared to our previous study using TbpB conjugated to Ctb (see Example 1). Within the four groups immunized IN with the chimeras, by day 35 the two groups immunized with the Ctb chimeras had antibody levels significantly different from the two groups immunized with the LtbIIb chimeras (P<0.05, day 35). However, s.c immunization with both Ctb and LtbIIb chimeras elicited antibody levels that were not significantly different from each other, or the IN immunized Ctb chimeras.

The antibody levels measured to the L2 domain of the chimeras were even lower than those measured to NB. By day 35 only the group immunized IN with NB-L2-Ctb had seroconverted. These levels were very low, in excess of 100 fold difference with the group immunized with the ThpA and TbpB-Ctb conjugates, as described in Example 1.

Because low serum antibody responses were detected after three IN immunizations, we boosted all the groups immunized with the chimeras IP. This vaccination strategy significantly enhanced antibody titers to TbpB in all groups immunized with the chimeras. These levels ranged from 50-100 fold greater than the antibody amounts measured before IP boost. Furthermore, the discrepancy observed in antibody levels measured to the chimeras compared to the previous study in the groups immunized with the TbpB-Ctb conjugates was decreased. Comparisons within the chimera groups following IP boost however still demonstrated a significant difference in antibody levels between the IN primed LtbIIb chimeras compared to rest of the chimeras.

In addition to enhancing antibody titers to TbpB, IP immunization increased antibodies against ThpA in all the groups immunized with the L2 chimeras. Though all groups immunized with the NB-L2 chimeras elicited ThpA specific antibodies, these levels were still much lower than the groups immunized with the conjugated full-length proteins as demonstrated in Example 1. Only the group immunized with NB-L2-LtbIIb (s.c./IP) surpassed 1 μg/mL in the serum on day 63. Comparison of all groups immunized with the chimeras on days 63 and 82 demonstrated a decline in ThpA specific antibody amounts.

### Vaginal Antibody Responses

Similar to what was observed in the sera, vaginal antibody levels in the groups immunized with the chimeras were much lower to TbpB prior to the IP boosts (Table 7). Prior to the IP boosts, only two groups had measurable TbpB specific IgA (Table 7, day 28). As anticipated, groups with the highest IgA levels were immunized with the Ctb chimeras. Vaginal IgG levels were non-existent in any of the chimera groups immunized IN, but were found in the group immunized Sc with the Ctb chimera (Table 7, day 28).

TABLE 7
Vaginal antibody levels specific for
TbpB detected on days 28 and 63^{a}

| | Day 28^{b} | | Day 63^{c} | |
|---|---|---|---|---|
| Immunization | IgA | IgG | IgA | IgG |
| NB-Ctb (IN/IP) | 0.4 x/÷ 4.4 | 0 | 0.5 x/÷ 3.0 | 7.1 x/÷ 3.4 |
| NB-L2-Ctb | <0.1^{e} | 0 | 0.6 x/÷ 5.3 | 10.8 x/÷ 1.7 |
| (IN/IP) | | | | |
| NB-LtbIIb | 0 | 0 | 0 | 0.2 x/÷ 4.7^{d} |
| (IN/IP) | | | | |
| NB-L2-LtbIIb | 0 | 0 | 0 | 0.5 x/÷ 4.9^{e} |
| (IN/IP) | | | | |
| NB-L2-Ctb | 0 | 5.8 x/÷ 3.5^{f} | 0.7 x/÷ 3.8^{g} | 12.8 x/÷ 1.3^{g} |
| (s.c./IP) | | | | |
| NB-L2-LtbIIb | 0 | 0 | <0.1^{e} | 1.2 x/÷ 3.4^{f} |
| (s.c./IP) | | | | |

^{a}Data are expressed as the geometric mean of the percentage of total corresponding antibody isotype concentration x/÷ standard deviation.
^{b}Day 28 is 7 days after final IN prime.
^{c}Day63 is 7 days after final boost.
^{d}Only one mouse had detectable TbpB-specific antibodies.
^{e}Only two mice had detectable TbpB-specific antibodies.
^{f}Only three mice had detectable TbpB-specific antibodies.
^{g}n = 4.

Following the IP boosts, most of the groups had measurable TbpB-specific IgA, and all of them had measurable TbpB-specific IgG (Table 7, day 63). Though IP immunization could elicit vaginal IgA, as most groups seroconverted, these levels were much lower compared to the groups immunized with the TbpB-Ctb conjugate (compare Table 7 with Example 1). Vaginal IgG levels to TbpB were much higher following IP immunization than the IgA levels. All mice had detectable TbpB specific IgG on day 63, which corresponded with their increase in serum IgG levels. Those mice with the highest serum IgG levels had the highest vaginal levels and vice versa (Table 7, day 63). This result is not unexpected as vaginal IgG levels are considered to result from serum transudation. In contrast to the situation with NB, we were unable to detect any ThpA-specific IgA or IgG in vaginal secretions of NB-L2 immunized mice.

### Serum Bactericidal Activity

In order to determine whether the chimeric antigens could elicit protective antibodies, we performed in vitro bactericidal assays using human sera as a complement source. We pooled day 63 sera from all groups and compared bactericidal activity. All the tested sera had similar bactericidal activity against the homologous strain FA19 (Table 8). Interestingly bactericidal activity against the heterologous strain MS 11 was 2 fold greater than against the homologous strain FA19. This difference could have been due in part to the greater sensitivity of MS11 to human complement (Carbonetti, N., V. Simnad, C. Elkins, and P. F. Sparling. 1990. Construction of isogenic gonococci with variable porin structure: effects on susceptibility to human serum and antibiotics. Mol. Microbiol. 4:1009-18). The other heterologous strain tested, FA1090, was the most resistant to bactericidal killing by all the sera tested. Surprisingly, the groups primed IN and s.c. with NB-L2-Ctb, had the highest bactericidal titers activity against this strain (Table 8). However, the group immunized with NB-Ctb did not elicit bactericidal antibodies detectable at the dilutions tested, even though it had the highest antibody titers against TbpB on day 63.

TABLE 8
Serum bactericidal activities of sera collected on day 63

| | Serum bactericidal titer^{a} for strain: | | | |
|---|---|---|---|---|
| Immunization Group | FA19 | MS11 | FA1090 | |
| NtermB-Ctb (IN/NP) | 200 (74% ± 9.2 | 400 (79% ± 1.4) | 25 | (<50)^{b} |
| NtermB-L2-Ctb | 200 (62% ± 0.7) | 400 (66% ± 21.2) | 5o | (60% ± 10.6) |
| (IN/IP) | | | | |
| NtermB-L2-Ctb | 200 (80% ± 8.5) | 400 (58% ± 9.9) | 100 | (70% ± 3.5) |
| (s.c./IP) | | | | |
| NtermB-L2bIIb | 200 (80% ± 1.4) | 400 (97% ± 2.1) | 25 | (<50)^{b} |
| (s.c./IP) | | | | |

^{a}Data are represented s the lowest reciprocal dilution that gave >50% killing. The average percent killing determined from duplicate assays ± standard deviation is shown in parentheses.
^{b}Assays conducted at 1/25 were performed only once and lower dilutions were not tested.

### Growth Inhibition Assays

Gonococcal strains were plated from freezer stocks onto GCB plates plus Kellogg's supplement I and 5 μM desferal to induce iron stress. Plates were incubated at 37° C. in a 5% CO₂ atmosphere for approximately 18 hours. Isolated colonies were removed from the plate and resuspended in iron free CDM. The absorbance was monitored at 600 nm until it reached 0.15. Following dilution, 87 μL was loaded into a sterile 96 well flat-bottomed microtiter plate. Iron saturated transferrin was then added to a final concentration of ˜7.5 μM (3 μL of 20 mg/mL iron saturated transferrin) and 10 μL of pooled vaginal wash was added. Following an initial absorbance reading at 600 nm in a microplate reader, the plates were incubated at 37° C. in a 5% CO₂ atmosphere with shaking at 225 rpm. Every two hours, the absorbance was determined as above. All samples were tested in duplicate, and each assay was conducted at least 3 separate times. Enumeration of the number of viable bacteria per well in a volume of 87 μl was determined to be 2×10⁷ and 8×10⁶ for FA19 and FA1090 respectively by using plate count methodology.

Having demonstrated bactericidal activity of serum derived antibodies, we decided to determine whether antibodies obtained from vaginal washes could impede the growth of the gonococcus in vitro. Using media containing transferrin as the sole iron source and pooled vaginal washes diluted 1/10, we were able to show inhibition of growth against the homologous strain (FA19) with washes from groups immunized with the NB chimera and the NB-L2 chimera. The vaginal washes did not show as much inhibition against the heterologous strain FA1090, but the NB-L2 wash samples were able to slow the growth of this strain. Because we were unable to measure vaginal antibodies against the L2 subunit in any of the vaginal washes, it is likely the inhibition of the NB-L2 immunized mice was due to the higher antibody concentrations measured in this group compared to the NB only immunized group (Table 9).

TABLE 9
Day 63 vaginal wash TbpB-specific antibody concentrations

Concentration (ng/mL)^{a}

| Immunization | IgA | IgG |
|---|---|---|
| NB-Ctb (IN/IP) | 188 ± 244 | 274 ± 303 |
| NB-L2-Ctb (IN/IP) | 307 ± 482 | 439 ± 352 |
| Control | 0 | 0 |

^{a}Data are represented as means ± standard deviation

### Discussion

This study has demonstrated the unique production of chimeric vaccines by genetically fusing epitopes of the gonococcal Tbps to the A2 subunit of cholera toxin and E. coli heat-labile enterotoxin IIb. We show that by genetically fusing Thp epitopes to the non-toxic A2 subunit, which natively and non-covalently binds to the non-toxic B subunit, we can form chimeric recombinant immunogens. We focused our efforts on two Tbp domains, the NB domain from TbpB and the L2 domain from ThpA. We were able to express and purify an NB-Ctb and an NB-LtbIIb chimera, as well as a double chimera consisting of the NB subunit in tandem with L2 in both Ctb and LtbIIb B subunits. Using this approach, we were able to elicit Thp-specific antibodies in mice following vaccination. The immunogenicity of these chimeric antigens however was relatively poor in comparison to the antibody amounts elicited to the full-length proteins using Ctb as an adjuvant (Example 1). After 3 priming vaccinations either IN or Sc, antibody responses were lower compared to our previous study using Ctb conjugated to full-length proteins (Example 1). However, after 2 IP boosts, serum and vaginal IgG antibody levels elicited to the NB portion of TbpB increased dramatically, especially with the Ctb chimeras. Vaginal IgA levels however were low for the Ctb chimeras, and non-existent with the LtbIIb chimeras. Although we could elicit robust serum and vaginal IgG to the NB portion of our chimeras, antibody responses to the L2 epitope of ThpA were poor in the sera and non-existent in the secretions, even after 5 vaccinations. The reasons for this difference in immunogenicity to each of these different Tbp domains are likely two-fold. First, there is a large size difference between these two domains. The NB domain has a predicted molecular weight of ˜44 kDa, while the L2 domain is ˜9 kDa. The small size of L2 could have accounted for its poor immunogenicity. Secondly, gonococcal TbpA is poorly immunogenic in comparison to TbpB as determined by relative antibody amounts following vaccination (Example 1). Thus, taking a small peptide from a protein that is already poorly immunogenic could account for the poor antibody response to L2. The rationale for combining the L2 domain with the NB domain was to determine whether combining a bigger more immunogenic polypeptide (NB) with a smaller less immunogenic peptide (L2) could enhance the immune response to the smaller peptide. Obviously in this study, combining L2 with NB in the manner described did not enhance antibody levels to L2, although the biological activities of the antibodies were augmented by the presence of L2 (see below).

As a correlate of protection, we performed in vitro bactericidal assays using human sera as a complement source to determine whether antibodies elicited to the NB or NB-L2 domains could be bactericidal. We have previously demonstrated the induction of cross-strain bactericidal antibodies following intranasal immunization using full-length ThpA and TbpB (Example 1). Here we demonstrate that regardless of the route of immunization (IN vs. s.c.), and the presence or absence of the L2 epitope, we were able to induce bactericidal titers against the homologous strain FA19. Against the heterologous strain MS11, we surprisingly induced similar bactericidal antibody titers using the chimeras as we did when using both full-length Tbps conjugated to Ctb (Example 1). Furthermore, the bactericidal titers elicited from the groups immunized with the chimeras were 2-fold higher than those against the homologous strain FA19.

Here we have established that by using defined domains from the transferrin binding proteins, we targeted the immune response to epitopes that may be sensitive to the bactericidal effects of antibodies.

Though vaginal ThpA antibodies were unmeasureable, we decided to test vaginal wash samples in vitro to see if antibodies from these washes could stop or slow the growth of the gonococcus using transferrin as the sole iron source. Against the homologous strain, FA19, we were able to completely stop the growth of this strain using both NB and NB-L2 wash samples. Against the heterologous strain FA1090 we were also able to slow the growth of this strain down compared to controls, but only with the NB-L2 samples. The ability to slow the growth of FA1090 was notable since the FA19 and FA1090 NB domain only share 57% identity. If we had been able to elicit L2 specific vaginal antibodies, we can speculate that growth inhibition would have been greater due to higher degree of identity between the L2 of FA19 and FA1090 (88%). Being able to slow the growth of both strains was an interesting discovery when the taking into account the concentrations of TbpB-specific antibodies in these washes. The concentrations of pooled TbpB-specific IgA and IgG in the NB-Ctb immunized group were 188 ng/mL and 274 ng/mL respectively. For the group immunized with the NB-L2-Ctb chimera, the pooled TbpB-specific IgA and IgG concentrations were 307 ng/mL and 439 ng/mL respectively. These samples were further diluted 1/10 for use in the growth inhibition assay. Furthermore, the number of bacteria used to initiate the assay was 2×10⁷ CFU for FA19 and 8×10⁶ CFU for FA1090. This suggests that if antibodies are elicited to the proper epitopes, high concentrations of Thp-specific antibody may not be needed to slow or stop the growth of the gonococcus in vivo.

### Example. 3

### Cocktails of Antigens

Cocktails of antigens are used to more broadly protect against neisserial diseases (gonorrhea and meningitis/meningococcemia). Thus, chimeric proteins that include a cholera toxin B subunit (or other mucosal adjuvant) fused to portions of the Tbps are constructed from a representative Neisseria gonorrhoeae strain and from a representative Neisseria meningitidis strain. These chimeric proteins are mixed to form a cocktail of protective antigens. Since characterized N. meningitidis strains fall into two broad classes, with respect to transferrin binding proteins, protection against all possible strains is accomplished by including representatives from both classes. Strain B16B6 is a representative of the “low molecular weight class”, which expresses relatively smaller, and more divergent, Tbps. Strain M982 is a representative of the “high molecular weight class”, which expresses larger Tbps. The high molecular weight Tbps from meningococcal strains (and from M982 in particular) are very similar to those of all of the N. gonorrhoeae strains characterized to date. A mixture of chimeric proteins from N. meningitidis (for example, strain B16B6) and from N. gonorrhoeae (for example, strain FA19) are combined into an immunogen cocktail, and the immune response generated is protective against infection by all Neisserial isolates.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. A fusion protein comprising
one or more transferrin binding proteins or antigenic regions thereof, and a mucosal adjuvant.

2. The fusion protein of claim 1, wherein said one or more transferrin binding proteins is a Neisseria transferrin binding protein.

3. The fusion protein of claim 2, wherein said one or more transferrin binding proteins originates from Neisseria gonorrhoeae.

4. The fusion protein of claim 3, wherein said one or more transferrin binding proteins is transferrin binding protein A or transferrin binding protein B.

5. The fusion protein of claim 1, wherein one or more antigenic regions includes L2 or NB, or both, from Neisseria gonorrhoeae.

6. The fusion protein of claim 2, wherein said one or more transferrin binding proteins originates from Neisseria meningitidis.

7. The fusion protein of claim 6, wherein said one or more transferrin binding proteins is transferrin binding protein A or transferrin binding protein B.

8. The fusion protein of claim 1, wherein said one or more antigenic regions includes L2 or NB, or both, from Neisseria meningitidis.

9. The fusion protein of claim 1, wherein said mucosal adjuvant originates from a toxin molecule.

10. The fusion protein of claim 9, wherein said mucosal adjuvant originates from Vibrio cholerae or Escherichia coli.

11. The fusion protein of claim 10, wherein said mucosal adjuvant is selected from the group consisting of B subunit of Vibrio cholerae toxin and B subunit of Escherichia coli heat labile toxin type I.

12. A method of eliciting antibodies to a transferrin binding protein in a mammal, comprising the step of
administering to said mammal a fusion protein comprising one or more transferrin binding proteins or antigenic regions thereof and a mucosal adjuvant, in an amount sufficient to elicit antibodies to said transferrin binding protein in said mammal.

13. The method of claim 12, wherein said step of administering is carried out intranasally.

14. The method of claim 12, wherein said antibodies are produced in one or more locations in said mammal selected from the group consisting of a urogenital tract, a oropharynx tract and serum.

15. The method of claim 12, wherein said antibodies include class IgA antibodies.

16. The method of claim 12, wherein said antibodies include bactericidal IgG antibodies.

17. The method of claim 12, wherein said one or more transferrin binding proteins or antigenic regions thereof originates from a Neisseria species.

18. The method of claim 17, wherein said Neisseria species is Neisseria gonorrhoeae.

19. The method of claim 18, wherein said one or more transferrin binding proteins is transferrin binding protein A or transferrin binding protein B.

20. The method of claim 17, wherein said one or more antigenic regions includes L2 or NB, or both, from Neisseria gonorrhoeae.

21. The method of claim 12, wherein said one or more transferrin binding proteins or antigenic regions thereof originates from Neisseria meningitidis.

22. The method of claim 21, wherein said one or more transferrin binding proteins is transferrin binding protein A or transferrin binding protein B.

23. The method of claim 21, wherein said one or more antigenic regions includes L2 or NB, or both, from Neisseria meningitidis.

24. The method of claim 12, wherein said mucosal adjuvant originates from a toxin molecule.

25. The method of claim 24, wherein said mucosal adjuvant originates from Vibrio cholerae or Escherichia coli.

26. The method of claim 25, wherein said mucosal adjuvant is selected from the group consisting of B subunit of Vibrio cholerae toxin and B subunit of Escherichia coli heat labile toxin type II.

27. An amino acid sequence as represented by SEQ ID NO: 8.

28. A nucleotide sequence encoding SEQ ID NO: 8.

29. A nucleotide sequence encoding a chimera comprising one or more Neisseia transferrin binding proteins or antigenic regions thereof and at least one mucosal adjuvant.

30. A method for inhibiting growth of Neisseria on a mucosal surface of a patient in need thereof, comprising the step of
administering to said patient a fusion protein or polypeptide, or antibodies thereto, said fusion protein or polypeptide comprising
one or more Neisseria transferrin binding proteins or antigenic regions thereof; and
a mucosal adjuvant.

31. The method of claim 30, wherein said one or more Neisseria transferrin binding proteins is transferrin binding protein A or transferrin binding protein B.

32. The method of claim 30, wherein said one or more antigenic regions includes L2 or NB, or both.
